(19) United States
(12) Patent Application Publication
(10) Pub. No.: US 2009/0036660 A1
(43) Pub. Date: Feb. 5, 2009

(54) Methods and compositions for generating mixtures of nucleic acid molecules

(76) Inventors: Joel Myerson, Berkeley, CA (US); Emily M. Leproust, Loveland, CO (US); Douglas J. Dellinger, Boulder, CO (US)

Correspondence Address: AGILENT TECHNOLOGIES INC., INTELLECTUAL PROPERTY ADMINISTRATION,LEGAL DEPT., MS BLDG. E P.O. BOX 7599, LOVELAND, CO, 80537, US

(21) Appl. No.: 11/831,771

(22) Filed: Jul. 31, 2007

(51) Int. Cl. C07H 21/00

(52) U.S. Cl. 536/23.1; 536/25.33

(57) ABSTRACT

In some embodiments, the present disclosure provides methods of making a mixture of nucleic acid molecules, the methods comprising the steps of: synthesizing on a substrate a population of nucleic acid molecules wherein each synthesized nucleic acid molecule comprises a substrate-attached proximal nucleic acid molecule, a distal nucleic acid molecule, and a cleavable linker linking the proximal nucleic acid molecule to the distal nucleic acid molecule, and harvesting distal nucleic acid molecules from the substrate by cleaving the cleavable linker under conditions that do not release the proximal nucleic acid molecule. Related compositions and kits are also provided.

### BACKGROUND

Known methods of fabricating biopolymer arrays include in situ synthesis methods or deposition of the previously obtained biopolymers. The in situ synthesis methods include those described in WO 98/41531 and the references cited therein for synthesizing polynucleotides. Such in situ synthesis methods can be basically regarded as iterating the sequence of: (a) depositing droplets of a protected monomer onto predetermined locations on a substrate to link with either a suitably activated substrate surface or with a previously deposited, deprotected monomer; (b) deprotecting the deposited monomer so that it can now react with a subsequently deposited protected monomer; and (c) depositing another protected monomer for linking. Different monomers may be deposited at different regions on the substrate during any one iteration so that the different regions of the completed array will have different desired biopolymer sequences. One or more intermediate further steps may be required in each iteration, such as oxidation and washing steps. The deposition methods basically involve depositing biopolymers at predetermined locations on a substrate which are suitably activated such that the biopolymers can link thereto. Biopolymers of different sequence may be deposited at different regions of the substrate to yield the completed array. Washing or other additional steps may also be used.

Large numbers of small amounts of individual polynucleotides can be synthesized in array format and cleaved off the surface (see, e.g., Tian, et al. (2004) Nature 432:1050 and Cuppoletti (WO2004059010)). There is a need for improved methods for preparing mixtures of polynucleotides.

### SUMMARY

In some embodiments, methods, compositions and kits for generating mixtures of nucleic acid molecules are provided. In some embodiments, the methods comprise:
a) synthesizing an array of surface-bound proximal nucleic acid molecules on a substrate,
b) incorporating a cleavable linker by contacting the proximal array of nucleic acid molecules with a cleavable phosphoramidite building block comprising:

R-Lc-Pr

wherein Pr is a hydroxyl protecting group,
Lc is a cleavable linker, and
R is a phosphoramidite group,
c) extending the building block to form distal nucleic acid molecules, and
d) cleaving the cleavable linker to release the distal nucleic acid molecules. The cleavable linker is cleaved under conditions which do not release the proximal nucleic acid molecules from the substrate surface. In some embodiments, the proximal nucleic acid is attached to the substrate by a non-cleavable attachment linkage.

Some embodiments of cleavable phosphoramidite building blocks are provided herein. Also provided are arrays employed in the subject methods and kits for practicing the subject methods.

Additional advantages and novel features of the methods, compositions, devices, and kits will be set forth in part in the description which follows, and in part will become apparent to those skilled in the art upon examination of the following description, or may be learned by practice of the methods.

### BRIEF DESCRIPTION OF THE DRAWINGS

FIG. 1 schematically shows some embodiments of methods for nucleic acid synthesis.

FIG. 2 schematically shows some embodiments of methods for nucleic acid synthesis.

FIG. 3 illustrates some embodiments of hydroxyl linkers.

FIG. 4 illustrates some embodiments of cleavable phosphoramidite building blocks.

FIG. 5 illustrates some embodiments of methods for nucleic acid synthesis.

FIG. 6 illustrates some embodiments of cleavable phosphoramidite building blocks.

FIG. 7 illustrates some embodiments of cleavable phosphoramidite building blocks.

FIG. 8 illustrates some embodiments of cleavable phosphoramidite building blocks.

### DESCRIPTION

Before describing the present disclosure in detail, it is to be understood that this disclosure is not limited to specific compositions, method steps, or kits, as such can vary. It is also to be understood that the terminology used herein is for the purpose of describing particular embodiments only, and is not intended to be limiting. Methods recited herein can be carried out in any order of the recited events that is logically possible, as well as the recited order of events. Where a range of values is provided, it is understood that each intervening value, between the upper and lower limit of that range, and any other stated or intervening value in that stated range, is encompassed within the description. The upper and lower limits of these smaller ranges may independently be included in the smaller ranges, and are also encompassed, subject to any specifically excluded limit in the stated range. Where the stated range includes one or both of the limits, ranges excluding either or both of those included limits are also included in the present disclosure. Also, it is contemplated that any optional feature of the disclosed variations described can be set forth and claimed independently, or in combination with any one or more of the features described herein.

All literature and similar materials cited in this application, including but not limited to patents, patent applications, articles, books, treatises, and internet web pages, regardless of the format of such literature and similar materials, are expressly incorporated by reference in their entirety for any purpose. In the event that one or more of the incorporated literature and similar materials differs from or contradicts this application, including but not limited to defined terms, term usage, described techniques, or the like, this application controls.

The practice of the present disclosure will employ, unless otherwise indicated, conventional techniques of synthetic organic chemistry, biochemistry, molecular biology, and the like, which are within the skill of the art. Such techniques are explained fully in the literature.

Unless specifically defined herein, all terms used herein have the same meaning as they would to one skilled in the art of the present disclosure. Practitioners are particularly directed to Sambrook et al. (1989) Molecular Cloning: A Laboratory Manual, 2^{nd} ed., Cold Spring Harbor Press, Plainview, N.Y., and Ausubel et al. (1999) Current Protocols in Molecular Biology (Supplement 47), John Wiley & Sons, New York, for definitions and terms of the art.

The publications discussed herein are provided solely for their disclosure prior to the filing date of the present application. Nothing herein is to be construed as an admission that the present disclosure is not entitled to antedate such publication by virtue of prior invention. Further, the dates of publication provided may be different from the actual publication dates, which may need to be independently confirmed.

As used in the specification and the appended claims, the singular forms “a,” “an” and “the” include plural referents unless the context clearly dictates otherwise. Thus, for example, reference to “a nucleic acid” includes a plurality of nucleic acids. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The term “comprising” does not exclude other elements or features. Also, elements described in association with different embodiments may be combined. “May” refers to optionally. “Optional” or “optionally” means that the subsequently described circumstance may or may not occur, so that the description includes instances where the circumstance occurs and instances where it does not.

Hyphens, or dashes, are used at various points throughout this specification to indicate attachment, e.g. where two named groups are immediately adjacent a dash in the text, this indicates the two named groups are attached to each other. Similarly, a series of named groups with dashes between each of the named groups in the text indicates the named groups are attached to each other in the order shown. Also, a single named group adjacent a dash in the text indicates the named group is typically attached to some other, unnamed group. In some embodiments, the attachment indicated by a dash may be, e.g. a covalent bond between the adjacent named groups. In some embodiments, the dash may indicate indirect attachment, i.e. with intervening groups between the named groups. At various points throughout the specification a group may be set forth in the text with or without an adjacent dash, (e.g. Lc, Lc- or -Lc-) where the context indicates the group is intended to be (or has the potential to be) bound to another group; in such cases, the identity of the group is denoted by the group name (whether or not there is an adjacent dash in the text). Note that where context indicates, a single group may be attached to more than one other group (e.g. where a linkage is intended, such as linking groups).

The present disclosure is based in part on the surprising discovery by applicant that when creating arrays of nucleic acids on surfaces, that the efficiency of the nucleic acid synthesis is increased after a certain number of cycles have been performed. Without wishing to be bound by theory, the use of a surface bound nucleic acid as described herein is believed to overcome undesirable surface effects that inhibit the nucleic acid synthesis chemistry.

In some embodiments, the disclosure concerns methods for generating mixtures of nucleic acid molecules. In some embodiments, the methods comprise:
a) synthesizing an array of surface-bound proximal nucleic acid molecules on a substrate,
b) incorporating a cleavable linker by contacting the proximal array of nucleic acid molecules with a cleavable phosphoramidite building block comprising:

R-Lc-Pr

wherein Pr is a hydroxyl protecting group,
Lc is a cleavable linker, and
R is a phosphoramidite group,
c) extending the building block to form distal nucleic acid molecules, and
d) selectively cleaving the cleavable linker to release the distal nucleic acid molecules.

A “cleavable activated phosphorus-containing building block” can be used as a starting point for nucleic acid synthesis. A cleavable activated phosphorus-containing building block, as described herein, can be incorporated using standard nucleic acid synthetic chemistry anywhere in a growing nucleic acid strand. Some embodiments of activated phosphorus-containing groups include phosphodiester, phosphotriester, phosphate triester, H-phosphonate and phosphoramidite groups. To facilitate description, and not by way of limitation, cleavable phosphoramidite building blocks will be primarily described herein.

A “cleavable phosphoramidite building block” can be used as a starting point for nucleic acid synthesis. A cleavable phosphoramidite building block, as described herein, can be incorporated using standard nucleic acid synthetic chemistry anywhere in a growing nucleic acid strand. A cleavable phosphoramidite building block for use in the present methods is selected such that the cleavable linker is not cleaved during the nucleic acid synthesis cycle. In some embodiments, as described herein, after synthesis of the distal nucleic acid is completed, the cleavable linker is cleaved. In some embodiments, the released distal nucleic acids each have a 3′ hydroxyl group. In some embodiments, the released distal nucleic acids each have a 3′ phosphate group and can be transformed into nucleic acids with a 3′-hydroxyl group (e.g., by chemical or enzymatic dephosphorylation).

In some embodiments, a cleavable activated phosphate building block comprises a “universal non-nucleoside building block” which can be used as a starting point for nucleic acid synthesis regardless of the nucleoside species at the 3′ end of the distal nucleic acid sequence. A “universal non-nucleoside building block” comprises a single activated phosphate (e.g., phosphoramidite) that will, after cleavage of the cleavable linker as described herein, result in a distal nucleic acid with any desired residue at the 3′-terminus. A universal non-nucleoside building block, as described herein, can be incorporated using standard nucleic acid synthetic chemistry anywhere in a growing nucleic acid strand. A universal non-nucleoside building block for use in the present methods can be selected such that the cleavable linker is not cleaved during the nucleic acid synthesis cycle. In some embodiments, as described herein, after synthesis of the distal nucleic acid is completed, the cleavable linker is cleaved. The cleavable linker can be selected such that cleavage of the attachment linkage does not occur (i.e., release of the proximal nucleic acid from the substrate does not occur) under those conditions which cleave the cleavable linker. In some embodiments, the released distal nucleic acids each have a 3′ hydroxyl group. In some embodiments, the released distal nucleic acids each have a 3′ phosphate group and can be transformed into nucleic acids with a 3′-hydroxyl group (e.g., by chemical or enzymatic dephosphorylation).

The cleavable linker may be any desired length and can be comprised of any suitable atoms that can include but not be limited to carbon, nitrogen, oxygen, sulfur and any combination thereof, as long as it functions in accordance with the present methods. The cleavable linker can comprise chemical groups, non-limiting examples of which include aliphatic bonds, double bonds, triple bonds, peptide bonds, aromatic rings, aliphatic rings, heterocyclic rings, ethers, esters, amides, and thioamides. The cleavable linker can form a rigid structure or be flexible in nature. In some embodiments, the cleavable linker may be of six or more atoms in length. Some embodiments of building blocks, which comprise cleavable linkers, are provided hereinbelow.

In some embodiments, R has the following structure:

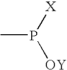

wherein X is —NQ¹Q² in which Q¹ and Q² may be the same or different and are typically selected from the group consisting of alkyl, aryl, aralkyl, alkaryl, cycloalkyl, alkenyl, cycloalkenyl, alkynyl, cycloalkynyl, optionally containing one or more nonhydrocarbyl linkages such as ether linkages, thioether linkages, oxo linkages, amine and imine linkages, and optionally substituted on one or more available carbon atoms with a nonhydrocarbyl substituent such as cyano, nitro, halo, or the like. In some embodiments, each of Y, Q¹ and Q² is independently a hydrocarbyl, substituted hydrocarbyl, heterocycle, substituted heterocycle, aryl or substituted aryl. In some embodiments, Y, Q¹ and Q² are selected from lower alkyls, lower aryls, and substituted lower alkyls and lower aryls (for example, substituted with structures containing up to 18, 16, 14, 12, 11, 10, 9, 8, 7, 6, 5, 4, 3 or 2 carbons). In some embodiments, Q¹ and Q² can have a total of from 2 to 12 carbon atoms. In some embodiments, Q¹ and Q² represent lower alkyl, and can be sterically hindered lower alkyls such as isopropyl, t-butyl, isobutyl, sec-butyl, neopentyl, tert-pentyl, isopentyl, sec-pentyl, and the like. In some embodiments, Q¹ and Q² both represent isopropyl. Q¹ and Q² are optionally cyclically connected. For example, Q¹ and Q² may be linked to form a mono- or polyheterocyclic ring having a total of from 1 to 3, usually 1 to 2 heteroatoms and from 1 to 3 rings. In such a case, Q¹ and Q² together with the nitrogen atom to which they are attached represent, for example, pyrrolidone, morpholino or piperidino. Non-limiting examples of —NQ¹Q² moieties include, but are not limited to, dimethylamine, diethylamine, diisopropylamine, dibutylamine, methylpropylamine, methylhexylamine, methylcyclopropylamine, ethylcyclohexylamine, methylbenzylamine, methylcyclohexylmethylamine, butylcyclohexylamine, morpholine, thiomorpholine, pyrrolidine, piperidine, 2,6-dimethylpiperidine, piperazine, and the like. In some embodiments, moiety “Y” is hydrido or hydrocarbyl, typically alkyl, alkenyl, aryl, aralkyl, or cycloalkyl. In some embodiments, Y represents: lower alkyl; electron-withdrawing β-substituted aliphatic, particularly electron-withdrawing β-substituted ethyl such as β-trihalomethyl ethyl, β-cyanoethyl, β-sulfoethyl, β-nitro-substituted ethyl, and the like; electron-withdrawing substituted phenyl, particularly halo-, sulfo-, cyano- or nitro-substituted phenyl; or electron-withdrawing substituted phenylethyl. In some embodiments, Y represents methyl, β-cyanoethyl, or 4-nitrophenylethyl. In some embodiments, Y is 2-cyanoethyl or methyl, and either or both of Q¹ and Q² is isopropyl.

In some embodiments, there are provided arrays comprising nucleic acids described by the following formula:

sm-Nucleic Acid₁-Lc-Nucleic Acid₂

wherein:
Lc is a cleavable linker as described herein;
Nucleic Acid₁ is a surface-bound proximal nucleic acid bound to the surface;
Nucleic Acid₂ is a distal nucleic acid bound to Nucleic Acid₁ via the cleavable linker; and
sm is a support medium.

In some embodiments, only the Nucleic Acid₂ differs between features of the array. In some embodiments, Nucleic Acid₁ is a single-stranded nucleic acid and may be oriented such that either the 3′ or 5′ end of the molecule is proximal to the substrate surface. In some embodiments, Nucleic Acid₂ is a single-stranded nucleic acid and may be oriented such that either the 3′ or 5′ end of the molecule is proximal to the substrate surface.

Nucleic Acid₁ can be chemically immobilized onto the surface of the medium by an attachment linkage that is orthogonal to the chemistry of the cleavable linker. In some embodiments, Nucleic Acid₁ is covalently attached by a non-cleavable attachment linkage. A non-cleavable attachment linker is devoid of a cleavable moiety. A non-cleavable attachment linkage is characterized in that there are no cleavage conditions that would allow release of the proximal nucleic acid without degrading the proximal nucleic acid.

An “internucleotide bond” refers to a chemical linkage between two nucleoside moieties, such as a phosphodiester linkage in nucleic acids found in nature, or such as linkages well known from the art of synthesis of nucleic acids and nucleic acid analogues. An internucleotide bond can include a phospho or phosphite group, and can include linkages where one or more oxygen atoms of the phospho or phosphite group are either modified with a substituent or replaced with another atom, e.g. a sulfur atom, or the nitrogen atom of a mono- or di-alkyl amino group.

A “pulse jet” is a device which can dispense drops in the formation of an array. Pulse jets operate by delivering a pulse of pressure to liquid adjacent an outlet or orifice such that a drop will be dispensed therefrom (for example, by a piezoelectric or thermoelectric element positioned in a same chamber as the orifice).

A “phospho” group includes a phosphodiester, phosphotriester, and H-phosphonate groups. In the case of either a phospho or phosphite group, a chemical moiety other than a substituted 5-membered furyl ring can be attached to O of the phospho or phosphite group which links between the furyl ring and the P atom.

A “protecting group” is used in the conventional chemical sense to reference a group, which reversibly renders unreactive a functional group under specified conditions of a desired reaction. After the desired reaction, protecting groups can be removed to deprotect the protected functional group. In some embodiments, protecting groups are removable (and hence, labile) under conditions which do not degrade a substantial proportion of the molecules being synthesized.

In some embodiments, hydroxyl groups can be protected with a “hydroxyl protecting group.” The term “hydroxyl protecting group compatible with nucleic acid synthesis” or “acid labile protecting moiety” or “removable protecting group” refers to a protecting group that can be used in nucleic acid synthesis as described herein. A wide variety of hydroxyl protecting groups can be employed in the methods of the disclosure. In general, protecting groups render chemical functionalities inert to specific reaction conditions, and can be appended to and removed from such functionalities in a molecule without substantially damaging the remainder of the molecule. Representative hydroxyl protecting groups are disclosed by Beaucage, et al., Tetrahedron 1992, 48, 2223 2311, and also in Greene and Wuts, Protective Groups in Organic Synthesis, Chapter 2, 2d ed, John Wiley & Sons, New York, 1991. Non-limiting examples of hydroxyl protecting groups include dimethoxytrityl (DMT), monomethoxytrityl, 9-phenylxanthen-9-yl (Pixyl) and 9-(p-methoxyphenyl)xanthen-9-yl (Mox), and/or trityl groups or other protecting groups. The hydroxyl protecting group can be removed from polynucleotide compounds of the disclosure by techniques well known in the art to form the free hydroxyl. In some embodiments, the protecting group is stable under basic conditions but can be removed under acidic conditions. For example, dimethoxytrityl protecting groups can be removed by protic acids such as formic acid, dichloroacetic acid, trichloroacetic acid, p-toluene sulphonic acid or with Lewis acids such as for example zinc bromide. (See for example, Greene and Wuts, supra.)

“Moiety” and “group” are used to refer to a portion of a molecule, typically having a particular functional or structural feature, e.g. a linking group (a portion of a molecule connecting two other portions of the molecule), or an ethyl moiety (a portion of a molecule with a structure closely related to ethane). A “moiety” or “group” includes both substituted and unsubstituted forms. Typical substituents include one or more lower alkyl, any halogen, hydroxy, or aryl, or optionally substituted on one or more available carbon atoms with a nonhydrocarbyl substituent such as cyano, nitro, halogen, hydroxyl, or the like.

“Bound” may be used herein to indicate direct or indirect attachment. In the context of chemical structures, “bound” (or “bonded”, or “bind”, or “binding”, or like term) may refer to the existence of a chemical bond directly joining two moieties or indirectly joining two moieties (e.g. via a linking group or any other intervening portion of the molecule). The chemical bond may be a covalent bond.

The term “functionalization” as used herein relates to modification of a solid substrate to provide a plurality of functional groups on the substrate surface. By a “functionalized surface” as used herein is meant a substrate surface that has been modified so that a plurality of functional groups are present thereon.

“Functionalized” references a process whereby a material is modified to have a specific moiety bound to the material, e.g. a molecule or substrate is modified to have the specific moiety; the material (e.g. molecule or support) that has been so modified is referred to as a functionalized material (e.g. functionalized molecule or functionalized support).

The term “nucleic acid” as used herein means a polymer composed of nucleotides, e.g. deoxyribonucleotides or ribonucleotides, or compounds produced synthetically (e.g. PNA as described in U.S. Pat. No. 5,948,902 and the references cited therein) which can hybridize with naturally occurring nucleic acids in a sequence specific manner analogous to that of two naturally occurring nucleic acids, e.g., can participate in Watson-Crick base pairing interactions.

The terms “ribonucleic acid” and “RNA” as used herein mean a polymer composed of ribonucleotides.

The terms “deoxyribonucleic acid” and “DNA” as used herein mean a polymer composed of deoxyribonucleotides.

The term “oligonucleotide” as used herein denotes single stranded nucleotide multimers of from about 10 to about 200 nucleotides in length.

The term “polynucleotide” as used herein refers to single or double stranded polymer composed of nucleotide monomers of generally greater than 100 nucleotides in length. As used herein, the phrase “predetermined nucleic acid sequence” means that the nucleic acid sequence of a nucleic acid molecule is known and was chosen before synthesis of the nucleic acid molecule.

The terms “nucleoside” and “nucleotide” are intended to include those moieties which contain not only the known purine and pyrimidine bases, but also other heterocyclic bases that have been modified. Such modifications include methylated purines or pyrimidines, acylated purines or pyrimidines, alkylated riboses or other heterocycles. In addition, the terms “nucleoside” and “nucleotide” include those moieties that contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like. Nucleotide sub-units of deoxyribonucleic acids are deoxyribonucleotides, and nucleotide sub-units of ribonucleic acids are ribonucleotides.

A “nucleotide monomer” refers to a molecule which is not incorporated in a larger oligo- or poly-nucleotide chain and which corresponds to a single nucleotide subunit; nucleotide monomers can also have activating or protecting groups, if such groups are necessary for the intended use of the nucleotide monomer.

A “polynucleotide intermediate” references a molecule occurring between steps in chemical synthesis of a polynucleotide, where the polynucleotide intermediate is subjected to further reactions to get the intended final product (e.g., a phosphite intermediate, which is oxidized to a phosphate in a later step in the synthesis), or a protected polynucleotide, which is then deprotected.

It will be appreciated that, as used herein, the terms “nucleoside” and “nucleotide” will include those moieties which contain not only the naturally occurring purine and pyrimidine bases, e.g., adenine (A), thymine (T), cytosine (C), guanine (G), or uracil (U), but also modified purine and pyrimidine bases and other heterocyclic bases which have been modified (these moieties are sometimes referred to herein, collectively, as “purine and pyrimidine bases and analogs thereof”). Such modifications include, e.g., methylated purines or pyrimidines, acylated purines or pyrimidines, and the like, or the addition of a protecting group such as acetyl, difluoroacetyl, trifluoroacetyl, isobutyryl, benzoyl, or the like. The purine or pyrimidine base can also be an analog of the foregoing; suitable analogs will be known to those skilled in the art and are described in the pertinent texts and literature. Common analogs include, but are not limited to, 1-methyladenine, 2-methyladenine, N6-methyladenine, N6-isopentyladenine, 2-methylthio-N6-isopentyladenine, N,N-dimethyladenine, 8-bromoadenine, 2-thiocytosine, 3-methylcytosine, 5-methylcytosine, 5-ethylcytosine, 4-acetylcytosine, 1-methylguanine, 2-methylguanine, 7-methylguanine, 2,2-dimethylguanine, 8-bromoguanine, 8-chloroguanine, 8-aminoguanine, 8-methylguanine, 8-thioguanine, 5-fluorouracil, 5-bromouracil, 5-chlorouracil, 5-iodouracil, 5-ethyluracil, 5-propyluracil, 5-methoxyuracil, 5-hydroxymethyluracil, 5-(carboxyhydroxymethyl)uracil, 5-(methylaminomethyl)uracil, 5-(carboxymethylaminomethyl)-uracil, 2-thiouracil, 5-methyl-2-thiouracil, 5-(2-bromovinyl)uracil, uracil-5-oxyacetic acid, uracil-5-oxyacetic acid methyl ester, pseudouracil, 1-methylpseudouracil, queosine, inosine, 1-methylinosine, hypoxanthine, xanthine, 2-aminopurine, 6-hydroxyaminopurine, 6-thiopurine and 2,6-diaminopurine.

As used herein, an “end” of a nucleic acid refers to the terminus of the nucleic acid, e.g., the last base or last chemical group at the 3′ or 5′ end of the nucleic acid.

The term “array” encompasses the term “microarray” and refers to an ordered array. Arrays, as described in greater detail below, are generally made up of a plurality of distinct or different features. The term “feature” is used interchangeably herein with the terms: “features,” “feature elements,” “spots,” “addressable regions,” “regions of different moieties,” “surface or substrate immobilized elements” and “array elements,” where each feature is made up of substrate immobilized nucleic acids. An array can include any one-dimensional, two-dimensional or substantially two-dimensional (as well as a three-dimensional) arrangement of addressable regions (i.e., features, e.g., in the form of spots) bearing nucleic acids, or synthetic mimetics thereof, and the like.

In some embodiments, a substrate may carry one, two, four or more arrays disposed on a front surface of the substrate. Depending upon the use, any or all of the arrays may be the same or different from one another and each may contain multiple spots or features. A typical array may contain one or more, including more than two, more than ten, more than one hundred, more than one thousand, more ten thousand features, or even more than one hundred thousand features, in an area of less than 20 cm² or even less than 10 cm², e.g., less than about 5 cm², including less than about 1 cm², less than about 1 mm², 100 μ², or even smaller. For example, features may have widths (that is, diameter, for a round spot) in the range from a 10 μm to 1.0 cm. In other embodiments each feature may have a width in the range of 1.0 μm to 1.0 mm, usually 5.0 μm to 500 μm, and more usually 10 μm to 200 μm. Non-round features may have area ranges equivalent to that of circular features with the foregoing width (diameter) ranges. At least some, or all, of the features are of different compositions (for example, when any repeats of each feature composition are excluded the remaining features may account for at least 5%, 10%, 20%, 50%, 95%, 99% or 100% of the total number of features).

In some embodiments of arrays, interfeature areas may be present which do not carry any polynucleotide. Such interfeature areas typically will be present where the arrays are formed by processes involving drop deposition of reagents but may not be present when, for example, light directed synthesis fabrication processes are used. It will be appreciated though, that the interfeature areas, when present, could be of various sizes and configurations. An array feature is generally homogenous in composition and concentration and the features may be separated by intervening spaces (although arrays without such separation can be fabricated).

As used herein, the term “essentially identical” as applied to synthesized nucleic acid molecules refers to nucleic acid molecules that are designed to have identical nucleic acid sequences, but that may occasionally contain minor sequence variations in comparison to a desired sequence due to base changes introduced during the nucleic acid molecule synthesis process, or due to other random processes. As used herein, essentially identical nucleic acid molecules are at least 95% identical to the desired sequence, such as at least 96%, such as at least 97%, such as at least 98%, such as at least 99% identical or absolutely identical, to the desired sequence.

As used herein, the term “complement” when used in connection with a nucleic acid molecule refers to the complementary nucleic acid sequence as determined by Watson-Crick base pairing. For example, the complement of the nucleic acid sequence 5′CCATG3′ is 5′CATGG3′.

A population of nucleic acid molecules can be synthesized on a substrate by any art-recognized means. The arrays employed in the subject methods may be generated de novo or obtained as a pre-made array from a commercial source, where in either case the array will have the characteristics described herein.

In some embodiments, an in situ method for fabricating a polynucleotide array using a functionalized support is as follows: at each of the multiple different addresses on a support at which features are to be formed, an iterative sequence is used in forming polynucleotides from nucleoside reagents. For example, the following attachment cycle at each feature to be formed can be used multiple times: (a) coupling an activated selected nucleoside (a monomeric unit) through a phosphite linkage to a functionalized support in the first iteration, or a nucleoside bound to the substrate (i.e. the nucleoside-modified substrate) in subsequent iterations; (b) optionally, blocking unreacted hydroxyl groups on the substrate bound nucleoside (sometimes referenced as “capping”); (c) oxidizing the phosphite linkage of step (a) to form a phosphate linkage; and (d) removing the protecting group (“deprotection”) from the now substrate bound nucleoside coupled in step (a), to generate a reactive site for the next cycle of these steps. The coupling can be performed by depositing drops of an activator and phosphoramidite at the specific desired feature locations for the array. In some embodiments, a final deprotection step is provided in which nitrogenous bases and phosphate group are simultaneously deprotected by treatment with ammonium hydroxide and/or methylamine under known conditions.

Different monomers may be deposited at different regions on the substrate during any one iteration so that the different regions of the completed array will have different desired biopolymer sequences. As indicated, one or more intermediate further steps may be required in each iteration, such as oxidation and washing steps.

Capping, oxidation and deprotection can be accomplished by treating the entire substrate (“flooding”) with a layer of the appropriate reagent. The functionalized support (in the first cycle) or deprotected coupled nucleoside (in subsequent cycles) provides a substrate bound moiety with a linking group for forming the phosphite linkage with a next nucleoside to be coupled in step (a). Final deprotection of nucleoside bases can be accomplished using alkaline conditions such as ammonium hydroxide, in another flooding procedure in a known manner. In some embodiments, a single pulse jet or other dispenser can be assigned to deposit a single monomeric unit.

Nucleic acid synthesis can be carried out by any art-recognized chemistry, including phosphodiester, phosphotriester, phosphate triester or N-phosphonate and phosphoramidite chemistries (see e.g., Froehler et al., Nucleic Acid Res 14:5399-5407, 1986; McBride et al., Tetrahedron Lett. 24:246-248, 1983). In some embodiments, methods of nucleic acid synthesis involve coupling an activated phosphorous derivative on the 3′ hydroxyl group of a nucleotide with the 5′ hydroxyl group of the nucleic acid molecule (see e.g., Gait (1984) Oligonucleotide Synthesis: A Practical Approach, IRL Press). Non-limiting embodiments of chemistry for the synthesis of polynucleotides are described in detail, for example, in Caruthers (1985) Science 230: 281-285; Itakura et al., Ann. Rev. Biochem. 53: 323-356; Hunkapillar et al. (1984) Nature 310: 105-110; and in “Synthesis of Oligonucleotide Derivatives in Design and Targeted Reaction of Oligonucleotide Derivatives”, CRC Press, Boca Raton, Fla., pages 100 et seq., U.S. Pat. No. 4,458,066, U.S. Pat. No. 4,500,707, Derivatives”, CRC Press, Boca Raton, Fla., pages 100 et seq., U.S. Pat. No. 4,458,066, U.S. Pat. No. 5,153,319, Derivatives”, CRC Press, Boca Raton, Fla., pages 100 et seq., U.S. Pat. No. 4,458,066, U.S. Pat. No. 5,869,643, European Patent Application EP 0294196, WO 9841531 and elsewhere. The synthesis can be carried out using combined oxidation/deprotection chemistry (see, e.g., Published U.S. Patent Application Nos. 20040230052 and 20020058802)

By way of example, a nucleotide monomer having an activated phosphoramidite group at the 3′ position, and a protected hydroxyl group at the 5′ position, reacts with a nucleic acid molecule, attached to a substrate, having a thiol or hydroxyl group at its 5′ position that is capable of forming a stable covalent bond with the phosphoramidite group at the 3′ position. Each coupling step adds one nucleotide to the end of the attached nucleic acid molecule. As described herein, after excess nucleotide monomer is washed away, a deprotection step reactivates the new end of the molecule for the next cycle (see, Blanchard et al., Biosensors & Bioelectronics (1996) 11:687-690.

Different monomers and activator may be deposited at different addresses on the substrate during any one cycle so that the different features of the completed array will have different desired biopolymer sequences. One or more intermediate further steps may be required in each cycle, such as the conventional oxidation, capping and washing steps in the case of in situ fabrication of polynucleotide arrays (again, these steps may be performed in a flooding procedure). In some embodiments, at least one additional step occurs between each cycle, such as oxidation of a phosphate bond to phosphate and deprotection of the 5′ (or 3′ in a reverse synthesis method) hydroxyl of a nucleoside phosphoramidite deposited and linked in a previous cycle.

In some embodiments, suitable nucleotides useful in the synthesis of nucleic acid molecules of the present methods include nucleotides that contain activated phosphorus-containing groups such as phosphodiester, phosphotriester, phosphate triester, H-phosphonate and phosphoramidite groups. In some embodiments, nucleic acid molecules can be synthesized using modified nucleotides, or nucleotide derivatives, such as for example, combinations of modified phosphodiester linkages such as phosphorothiate, phosphorodithioate and methylphosphonate, as well as nucleotides having modified bases such as inosine, 5′-nitroindole and 3′ nitropyrrole. Additionally, it is possible to vary the charge on the phosphate backbone of the nucleic acid molecule, for example, by thiolation or methylation, or to use a peptide rather than a phosphate backbone. The making of such modifications is within the skill of one trained in the art.

Synthesis of nucleic acid molecules comprising RNA can similarly be accomplished using the present methods. A range of modifications can be introduced into the base, the sugar, or the phosphate portions of oligoribonucleotides, e.g., by preparation of appropriately protected phosphoramidite or H-phosphonate ribonucleoside monomers, and/or coupling such modified forms into oligoribonucleotides by solid-phase synthesis. Modified ribonucleoside analogues include, for example, 2′O-methyl, 2′-O-allyl, 2′-fluoro, 2′-amino phosphorothioate,2′-O-Me methylphosphonate, 5′-O-Silyl-2′-O-ACE, 2′-O-TOM, alpha-ribose and2′-5′-linked ribonucleoside analogs.

In some embodiments of the present methods, nucleic acid molecules are synthesized on a surface of a substrate, such as a flat substrate, which may be textured or treated to increase surface area. The substrate may comprise a membrane, sheet, rod, tube, cylinder, bead or other structure. In some embodiments, the substrate comprises a non-porous medium, such as a planar glass substrate. The surface of the substrate typically has, or can be chemically modified to have, reactive groups suitable for attaching organic molecules. Examples of such substrates include, but are not limited to, glass, silica, silicon, plastic, (e.g., polypropylene, polystyrene, Teflon™, polyethylimine, nylon, polyester), polyacrylamide, fiberglass, nitrocellulose, cellulose acetate, or other suitable materials. The substrate may be treated in such a way as to enhance the attachment of nucleic acid molecules. For example, a glass substrate may be treated with polylysine or silane to facilitate attachment of nucleic acid molecules. Silanization of glass surfaces for oligonucleotide applications has been described (see, Halliwell et al. (2001) Anal. Chem. 73:2476-2483). In some embodiments, the surface of the substrate to which nucleic acid molecules are attached bears chemically reactive groups, such as carboxyl, amino, hydroxyl and the like (e.g., Si—OH functionalities, such as are found on silica surfaces).

In some embodiments of the methods, an attachment linkage is attached to the substrate and a proximal nucleic acid molecule is then synthesized at a chemically reactive group of the attachment linkage. Examples of useful attachment linkages include, for example, silane, aryl acetylene, ethylene glycol, hydroxyl. diamines, diacids, amino acids, or combinations thereof. The attachment linkages may be attached to the substrate via carbon-carbon bonds using, for example, (poly)trifluorochloroethylene surfaces, or, for example, by siloxane bonds to glass or silicon oxide surfaces. Methods of silanization of glass surfaces for oligonucleotide attachment are further described in Halliwell et al., Anal. Chem. (2001) 73:2476-2483.

In some embodiments, a solid support, such as glass is reacted with a silanol linker to provide an attachment point for synthesis of an oligonucleotide at a location on the solid support, to thereby form a feature comprising at least one oligonucleotide at the location. For example, a linker can be attached to the support and a chemically active attachment point or functional group (such as a hydroxyl group, for example) can be generated (i.e., generating a functionalized support) for bonding to a deposited monomer. (See, e.g., as described in U.S. Pat. No. 6,444,268, published U.S. Pat. Application No. 20030186226, and in Southern, E. M., Maskos, U. and Elder, J. K. (1992) Genomics 13:1007-1017.) The attachment linkages may be attached, for example, in an ordered array. In some embodiments, the attachment linkages may be provided with a functional group to which is bound a protective group, such as a photolabile protecting group. In some embodiments, the attachment linkages contain a photocleavable spacer such as photocleavable spacer phosphoramidite monomers (available from Glen Research, 22825 Davis Drive, Sterling, Va. 20164) which can be synthesized on a silanized glass substrate with hydroxyl functionality.

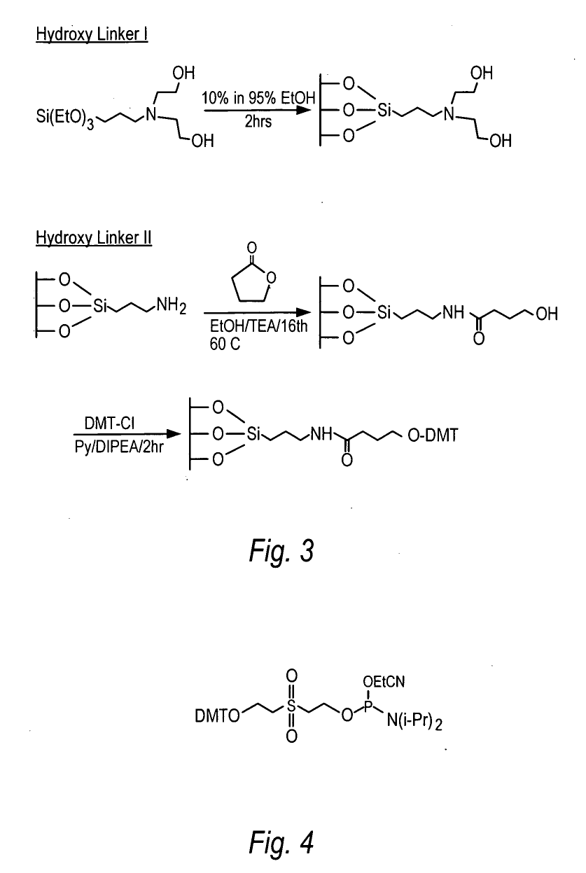

As mentioned above, proximal nucleic acids present on the substrate (e.g. at a feature of the array) can be bound to the substrate via a cleavable or via a non-cleavable attachment. The attachment may comprise a non-cleavable linkage, non-limiting examples of which are shown in FIG. 3. Non-limiting examples of non-cleavable attachment linkages are also described in U.S. Pat. No. 6,444,268. In some embodiments, a non-cleavable linker is devoid of a cleavable site (i.e., is devoid of a cleavable moiety). In some embodiments, a non-cleavable linker is devoid of a chemically cleavable site or a photolabile site. Non-limiting examples of chemically cleavable sites include an ester, succinate, urethane, benzyl alcohol derivatives, acetals, thioactelas, or sulfonyl.

For cleavable attachment linkers, the attachment may be cleavable by a number of different mechanisms. In certain embodiments, the attachment linker may be cleaved by light, i.e. photocleavable, or the attachment linker may be chemically cleavable, e.g., acid- or base-labile. In some embodiments, the attachment linker comprises either a photocleavable moiety or chemically cleavable moiety. Photocleavable or photolabile moieties that may be employed include, but are not limited to: o-nitroarylmethine and arylaroylmethine, as well as derivatives thereof, and the like.

In some embodiments, predetermined nucleic acid sequences are synthesized on a substrate, to form a high density microarray, by means of an ink jet printing device for oligonucleotide synthesis, such as described by Blanchard in U.S. Pat. No. 6,028,189; Blanchard et al. (1996) Biosensors and Bioelectrics 11:687-690; Blanchard, Synthetic DNA Arrays in Genetic Engineering, Vol. 20, J. K. Setlow, Ed. Plenum Press, New York at pages 111-123; U.S. Pat. Nos. 6,028,189; 6,242,266; 6,232,072; 6,180,351; 6,171,797; 6,323,043; and U.S. patent application Ser. No. 09/302,898 filed Apr. 30, 1999. The nucleic acid sequences in such microarrays can be synthesized in arrays, for example on a glass slide, by serially depositing individual nucleotide bases in “microdroplets” of a high surface tension solvent such as propylene carbonate. The microdroplets have small volumes (e.g., 100 picoliters (pL) or less, or 50 pL or less) and are separated from each other on the microarray (e.g., by hydrophobic domains) to form surface tension wells which define the areas containing the array elements (i.e., the different populations of nucleic acid. molecules). In some embodiments, microarrays manufactured by this ink-jet method are of high density. In some embodiments, the arrays have a density of at least about 2,000 different nucleic acid molecules per 1 cm². The proximal nucleic acid molecules may be covalently attached directly to the substrate, or to an attachment linkage to the substrate at either the 3′ or 5′ end of the proximal nucleic acid.

Exemplary ink jet printing devices suitable for oligonucleotide synthesis in the practice of the present methods contain microfabricated ink-jet pumps, or nozzles, which are used to deliver specified volumes of synthesis reagents to an array of surface tension wells (see, Kyser et al. (1981) J. Appl. Photographic Eng. 7:73-79). The pumps can be made, for example, by using etching techniques known to those skilled in the art to fabricate a shallow cavity and channels in silicon. A thin glass membrane is then anodically bonded to the silicon to seal the etched cavity, thus forming a small reservoir with narrow inlet and exit channels. When the inlet end of the pump is dipped in the reagent solution, capillary action draws the liquid into the cavity until it comes to the end of the exit channel. When an electrical pulse is applied to the piezoelectric element glued to the glass membrane it bows inward, ejecting a droplet out of the orifice at the end of the pump. For oligonucleotide synthesis in two dimensional arrays, pumps that deliver 100 pL droplets or less on demand at rates of several hundred Hertz (Hz) are applicable. However, the droplet volume or speed of the pump can vary depending on the need. For example, if a larger array is to be synthesized with the same surface area, then smaller droplets can be dispensed. Additionally, if synthesis time is to be decreased, then operation speed can be increased. Such parameters are known to those skilled in the art and can be adjusted as needed (see, e.g., U.S. Pat. Nos. 6,028,189; 6,375,903; and 7,072,500).

The present disclosure is not limited to pulse jet type deposition systems. Other drop deposition methods can be used for fabrication, such as are known in the art. Also, instead of drop deposition methods, photolithographic array fabrication methods may be used. In particular, any type of array fabricating apparatus can be used to contact the substrate with nucleotide monomers, including those such as described in U.S. Pat. No. 5,807,522, or an apparatus that can employ photolithographic techniques for forming arrays of moieties, such as described in U.S. Pat. No. 5,143,854 and U.S. Pat. No. 5,405,783, or any other suitable apparatus which can be used for fabricating arrays. For example, robotic devices for precisely depositing aqueous volumes onto discrete locations of a support surface, i.e., arrayers, are also commercially available from a number of vendors, including: Genetic Microsystems; Cartesian Technologies; Beecher Instruments; Genomic Solutions; and BioRobotics. Other methods and apparatus are described in U.S. Pat. Nos. 4,877,745; 5,338,688; 5,474,796; 5,449,754; 5,658,802; and 5,700,637. Patents and patent applications describing arrays of biopolymeric compounds and methods for their fabrication include: U.S. Pat. Nos. 5,242,974; 5,384,261; 5,405,783; 5,412,087; 5,424,186; 5,429,807; 5,436,327; 5,445,934; 5,472,672; 5,527,681; 5,529,756; 5,545,531; 5,554,501; 5,556,752; 5,561,071; 5,599,695; 5,624,711; 5,639,603; 5,658,734; WO 93/17126; WO 95/11995; WO 95/35505, WO 97/14706, WO 98/30575; EP 742 287; and EP 799 897. See also Beier et al. (1999) “Versatile derivatisation of solid support media for covalent bonding on DNA-microchips”, Nucleic Acids Research 27:1970-1977. (See also, Green et al., Curr. Opin. in Chem. Biol. 2:404-410 (1998), Gerhold et al, TIBS, 24:168-173 (1999), U.S. Pat. Nos. 6,090,995, 6,030,782, 5,700,637, 6,054,270, 5,919,626, 5,858,653, 5,837,832, 5,744,305, 5,445,934, WO99/58708, and Singh-Gasson et al., Nature Biotechnology 17:974-978 (1999)).

As described above, a plurality of nucleic acid molecules can be synthesized to form a high-density microarray. A nucleic acid microarray, or chip, is an array of nucleic acid molecules, such as synthetic oligonucleotides, disposed in a defined pattern onto defined areas of a solid support (see, Schena, BioEssays 18:427, 1996). The arrays are reproducible, allowing multiple copies of a given array to be produced and easily compared with each other. Microarrays can be made from materials that are stable under nucleic acid synthesis and cleavage conditions as described herein. In some embodiments, the nucleic acid molecules on the array are single-stranded nucleic acid sequences.

In some embodiments, the array is a positionally addressable array in that each nucleic acid molecule of the array is localized to a known, defined area on the substrate such that the identity (i.e., the sequence) of each nucleic acid molecule can be determined from its position on the array (i.e., on the substrate surface). For example, a substrate may have at least from about 1,000 to about 30,000, from about 1,000 to about 1,000,000, or more, separate defined areas. The size of each defined area on a substrate can be chosen to allow for efficient cleavage of the cleavable linker, as described herein, and thus release of the distal nucleic acids. For example, in some embodiments, approximately 0.3 fmole of distal nucleic acid is synthesized per defined area.

The proximal nucleic acid may be oriented such that either the 3′ or 5′ end of the molecule is proximal to the substrate surface, e.g., by controlling the synthesis reaction. Exemplary chain lengths of the synthesized proximal nucleic acid molecules can be in the range of about 2 to about 15 nt in length, about 2 to about 100 nt in length, about 2 to about 1000, or more, nt in length.

As described herein, cleavage of distal nucleic acids from an array can be used to produce a plurality of solution phase nucleic acids. For each feature present on the template array, there is at least one nucleic acid in the plurality that corresponds to the feature.

As indicated herein, the distal nucleic acids on a precursor array have sequences that can be chosen based on the particular application in which the array is to be used, and specifically the intended use of nucleic acids that are released from the array substrate.

In some aspects, the plurality of nucleic acids released from the array have a known composition. By known composition is meant that, because of the way in which the plurality is produced, the sequence of each distinct nucleic acid in the plurality can be predicted with a high degree of confidence. In some embodiments, the relative amount or copy number of each distinct nucleic acid of differing sequence in the plurality also is known. For example, the plurality of nucleic acids may be known to include a constituent distal nucleic acid corresponding to each feature of the precursor array used to produce it, such that each feature of the precursor array is represented in the plurality nucleic acids released from the array.

The amounts of each distinct nucleic acid in the plurality may be equimolar or non-equimolar, and can be conveniently chosen and controlled by employing a precursor array with the desired number of features (as well as molecules per feature) for each member of the plurality. For example, where a plurality of released nucleic acids having equimolar amounts of member nucleic acids is desired, a precursor array with the same number of features for each member distal nucleic acid is employed. Alternatively, where a plurality of released nucleic acids is desired in which there are twice as many nucleic acids of a first sequence as compared to a second sequence, a precursor array that has two times as many features comprising distal nucleic acids of the first sequence as compared to the second sequence may be employed.

The number of different or distinct nucleic acids of differing sequence present in a plurality of released nucleic acids can vary, but is generally at least about 2, at least about 5, at least about 10, such as at least about 20, at least about 50, at least about 100 or more, where the number may be as great as about 1000, about 5000, about 25,000, about 50,000, about 100,000, about 1,000,000, or greater. Any two given nucleic acids in the product pluralities are considered distinct or different if they include a stretch of at least 20 nucleotides in length in which the sequence similarity is less than 100%, less then 98%, less than about 80%, less than about 75%, or about 60%, as determined using a suitable program (using default settings) known in the art, e.g., such as FASTA or BLASTN (see, e.g., ncbi.nlm.nih.gov for information about default parameters). Alignment may also be performed manually by inspection.

Nucleic acids released from an array can comprise a heterogeneous mixture or a set of individual homogeneous nucleic acid compositions, depending on intended use.

Populations of released nucleic acids can remain mixed or can be sorted in one or more further processing steps, e.g., such as by binding to complementary nucleic acids bound to a solid support.

In those embodiments where the plurality of released nucleic acids comprise a set of homogenous nucleic acid populations, the constituent members of the set can be, in some aspects, physically separated, such as present on different locations of a solid support (e.g., of the precursor array), present in different containment structures, and the like.

In some embodiments, the present disclosure is also directed to selectably cleavable sites which are cleavable using chemical reagents. The cleavable sites can be created by incorporation of cleavable linkers into polynucleotide chains as described herein. Cleavage of distal nucleic acids at features on the array can be used to produce a solution phase mixture of nucleic acids. Generally, the cleavable step comprises contacting the array with an effective amount of a cleavage agent and/or exposing the array to a suitable cleavage condition. A cleavable linker may be cleaved by a number of different mechanisms. The cleavage agent and/or condition can be chosen in view of the particular nature of the cleavable linker that is to be cleaved, such that the linker is labile, and such that the attachment linker (or attachment means) is stable, with respect to the chosen cleavage agent and/or condition.

As described herein, following provision of a precursor array, a next step may include cleaving the cleavable linker to produce a solution phase mixture or population of nucleic acids. The distal nucleic acid molecules can be harvested from the substrate by any useful means. The array can be subjected to cleavage conditions sufficient to cleave the cleavable linker but which are not sufficient to release the surface bound nucleic acids from the substrate surface. As described above, this step can comprise contacting the array with an effective amount of a cleavage agent. The array can be contacted with a chemical capable of selectively cleaving the cleavable linker.

In the cleavage step, the array can be contacted with a chemical capable of cleaving the cleavable linker, e.g., an appropriate acid, base oxidant, or reducer, depending on the nature of the cleavable linker. In some embodiments, cleavable linkers comprise the following: base-cleavable sites such as esters, (cleavable by, for example, ammonia, methylamine, trimethylamine, or sodium hydroxide) (such as, e.g., succinates), quaternary ammonium salts (cleavable by, for example, diisopropylamine) and urethanes (cleavable by aqueous sodium hydroxide); acid-cleavable sites such as benzyl alcohol derivatives (cleavable using trifluoroacetic acid), teicoplanin aglycone (cleavable by trifluoroacetic acid followed by base), acetals and thioacetals (also cleavable by trifluoroacetic acid), thioethers (cleavable, for example, by HF or cresol) and sulfonyls (cleavable by trifluoromethane sulfonic acid, trifluoroacetic acid, thioanisole, or the like); nucleophile-cleavable sites such as phthalamide (cleavable by substituted hydrazines), esters (cleavable by, for example, aluminum trichloride); and Weinreb amide (cleavable by lithium aluminum hydride); and other types of chemically cleavable sites, including phosphorothioate (cleavable by silver or mercuric ions) and diisopropyldialkoxysilyl (cleavable by fluoride ions). Non-limiting examples of cleavable sites include: dialkoxysilane, β-cyano ether, amino carbamate, dithoacetal, disulfide, as well as derivatives thereof and the like. Other cleavable sites will be apparent to those skilled in the art or are described in the pertinent literature and texts (e.g., Brown (1997) Contemporary Organic Synthesis 4:216-237). In some embodiments, the cleavable linker comprises an ester bond which is susceptible to hydrolysis by exposure to a hydrolyzing agent, such as hydroxide ions (e.g., an aqueous solution of sodium hydroxide or ammonium hydroxide).

In some embodiments, the cleavage agent is a basic solution. Basic solutions of interest for use in the subject methods are any solutions that include a base and are sufficiently strong such that when contacted with the surface of the substrate, the desired fluid cleavage product that contains solution phase nucleic acids is produced. In some embodiments, the basic solution employed as the cleavage agent is a solution having a pH from about 8 to about 14, such as from about 9 to about 13, and including from about 10 to about 12. In some embodiments, the basic salt of the basic solution may be one having a pKₐ that ranges from about 8 to about 16, such as from about 9 to about 14, and including from about 10 to about 12. The concentration of the base in the solution may vary, but in some embodiments ranges from about 0.1 M to about 9 M, such as from about 0.8 M to about 8.5 M. Representative solutions of interest as cleavage agents for use in the subject methods include, but are not limited to, solutions of ammonia, methylamine, ethylamine and the like for basic solutions and Bu₄NF in THF, Pyridine/HF in THF, HF in Acetonitrile, SiF₄ in Acetonitrile, H₂SiF₆/TEA in acetonitrile and the like for acid hydrolysis cleavage, where in some embodiments, the solution is an ammonia solution.

The chemical cleavage agent is contacted with the substrate for a period of time sufficient for the distal nucleic acids to be released from the surface of the support. In some embodiments, contact is maintained for a period of time ranging from about 0.5 h to about 144 h, such as from about 2 h to about 120 h, and including from about 4 h to about 72 h. Any convenient method may be used to contact the cleavage agent with the nucleic acid displaying substrate. For instance, contacting may include, but is not limited to: submerging, flooding, rinsing, spraying, etc. Contact may be carried out at any convenient temperature, where in representative embodiments contact is carried out at temperatures ranging from about 0 C.° to about 60 C.°, including from about 20 C.° to about 40 C.°, such as from about 20 C.° to about 30 C.°.

In some embodiments, a cleavable linker comprises a nucleotide cleavable by an enzyme such as nucleases, glycosylases, among others. A wide range of polynucleotide bases may be removed by DNA glycosylases, which cleaves the N-glycosylic bond between the base and deoxyribose, thus leaving an abasic site (see, e.g., Krokan et. al. (1997) Biochem. J. 325:1-16). The abasic site in a polynucleotide may then be cleaved by Endonuclease IV, leaving a free 3′-OH end. Suitable DNA glycosylases may include uracil-DNA glycosylases, G/T(U) mismatch DNA glycosylases, alkylbase-DNA glycosylases, 5-methylcytosine DNA glycosylases, adenine-specific mismatch-DNA glycosylases, oxidized pyrimidine-specific DNA glycosylases, oxidized purine-specific DNA glycosylases, EndoVIII, EndoIX, hydroxymethyl DNA glycosylases, formyluracil-DNA glycosylases, pyrimidine-dimer DNA glycosylases, among others. Cleavable base analogs that are readily available synthetically. In some embodiments, a uracil may be synthetically incorporated in a polynucletide to replace a thymine, where the uracil is the cleavage site and site-specifically removed by treatment with uracil DNA glycosylase (see, e.g., Kunkel, T. A. (1985) Proc. Natl. Acad. Sci. USA 82:488-492; Lindahl (1990) Mutat. Res. 238:305-311; Published U.S. Patent Application No. 20050208538). The uracil DNA glycosylases may be from viral or plant sources, and is available commercially (e.g., Invitrogen, Catalogue no. 18054-015). The abasic site on the polynucleotide strand may then be cleaved by E. coli Endonuclease IV.

The distal nucleic acid molecules can be harvested from the substrate by any useful means. To release the distal nucleic acids, in some embodiments, the entire substrate can be treated with cleavage agent (e.g., hydrolyzing agent), or alternatively, a cleavage agent can be applied to a portion of the substrate.

In some embodiments, the cleavage conditions, as described herein, are also effective to cause base deprotection of the nucleic acids (e.g. removal of protecting groups from the heterocyclic bases of the nucleotide subunits) and/or phosphate deprotection (removal of the phosphate protecting groups). Thus, base or phosphate deprotection can occur concurrently with the cleavage reaction. In other embodiments, base or phosphate deprotection can occur prior to the cleavage reaction. This allows convenient removal of the base or phosphate protecting groups and washing of the array to remove the deprotection products before cleavage of the distal nucleic acids from the surface. In some embodiments, base or phosphate deprotection may occur after cleavage of the distal nucleic acids from the surface of the substrate. Base and/or phosphate deprotection can be accomplished by contacting the base (and/or phosphate)-protected nucleic acids with a deprotection agent. Deprotection of nucleic acids and the deprotection agents used are well known and need not be described further here.

The above-described cleavage methods result in the production of a plurality of solution phase nucleic acids. For each feature present on the template array, there is at least one nucleic acid in the product plurality that corresponds to the feature, whereby corresponds is meant that the nucleic acid is one that is generated by cleavage of the cleavable linker of the feature of the array. In some embodiments, the length of each of the released nucleic acids present in the resultant plurality ranges from about 10 to about 1000 nt, such as from about 20 to about 500 nt, including from about 30 to about 120 nt.

In some embodiments, the plurality of nucleic acids produced in some embodiments of the subject methods is characterized by having a known composition. By known composition is meant that, because of the way in which the plurality is produced, the sequence of each distinct nucleic acid in the product plurality can be predicted with a high degree of confidence. Accordingly, the sequence of each individual or distinct nucleic acid in the product plurality is known. In some embodiments, the relative amount or copy number of each distinct nucleic acid of differing sequence in the plurality is known.

In some embodiments, the amount or copy number of each distinct nucleic acid of differing sequence in the product plurality is known. The amounts of each distinct nucleic acid in the product plurality may be equimolar or non-equimolar, and can be conveniently chosen and controlled by employing a precursor array with the desired number of features (as well as molecules per/feature) for each member of the plurality. For example, where a product plurality that is equimolar for each member nucleic acid is desired, an array with the same number of features for each member nucleic acid is employed. Alternatively, where a product plurality is desired in which there are twice as many nucleic acids of one sequence as compared to another sequence, an array that has two times as many features of the one sequence as compared to the another sequence may be employed.

In some embodiments, the nucleic acids of the product pluralities are single-stranded ribonucleic acids. When the product nucleic acids of the plurality are single-stranded, they may be linear or assume some secondary configuration, e.g., a hairpin configuration, and the like.

The product plurality of nucleic acids may be a heterogeneous mixture or a set of individual homogeneous nucleic acid compositions, depending on the intended use of the product plurality.

The product pluralities of nucleic acids can be physically separated from the substrate as part of or following the cleavage step, as described herein. As such, the product of the cleavage step is a solution phase mixture of nucleic acids.

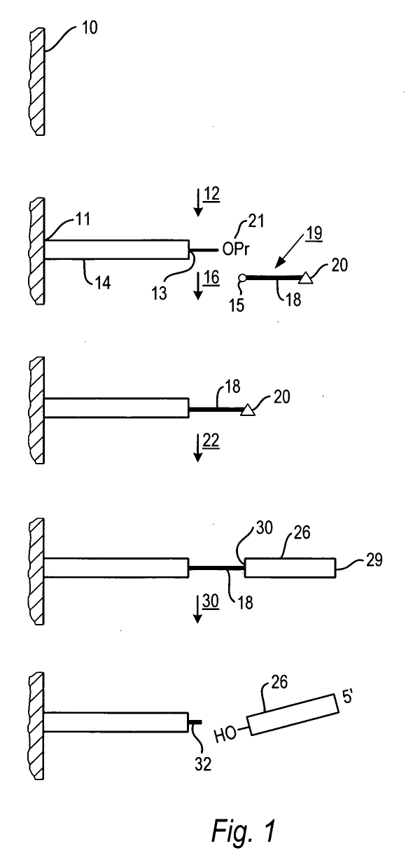

In accordance with some embodiments of the present methods, FIG. 1 shows a representative proximal nucleic acid molecule 14 synthesized on a substrate 10 (although it will be understood that, in the practice of the present methods, numerous nucleic acid molecules 14 are simultaneously synthesized on substrate 10). At step 12, nucleic acid molecule 14 is synthesized, as described herein, on substrate 10. Nucleic acid molecule 14 includes a 5′ end 13 and a 3′ end 11 which is covalently attached to the substrate. Step 16 includes removal of hydroxyl protecting group 21, and the resulting 5′ terminal hydroxyl of nucleic acid molecule 14 is then reacted with phosphoramidite group 15 of a a cleavable phosphoramidite building block 19, as described herein, to generate a linkage 18. Building block 19 includes protected hydroxyl group 20. At step 22, distal nucleic acid molecule 26 is synthesized, according to methods described herein, and includes a 3′ end 31, and a 5′ end 29. At step 30, the substrate is exposed to conditions that effect cleavage of linker 18 with no release, or essentially no release, of nucleic acid 14 from the substrate 10. In the embodiment shown, a free 3′ hydroxyl is generated in nucleic acid molecule 26. A portion 32 of linker 18 remains attached to nucleic acid molecule 14.

In some embodiments, a portion 32 does not remain attached to the proximal nucleic acid 14 during cleavage step 30 (not shown). In some embodiments, a portion of linker 18 can remain attached to nucleic acid 26, but can be released upon further exposure to a cleavage agent (not shown).

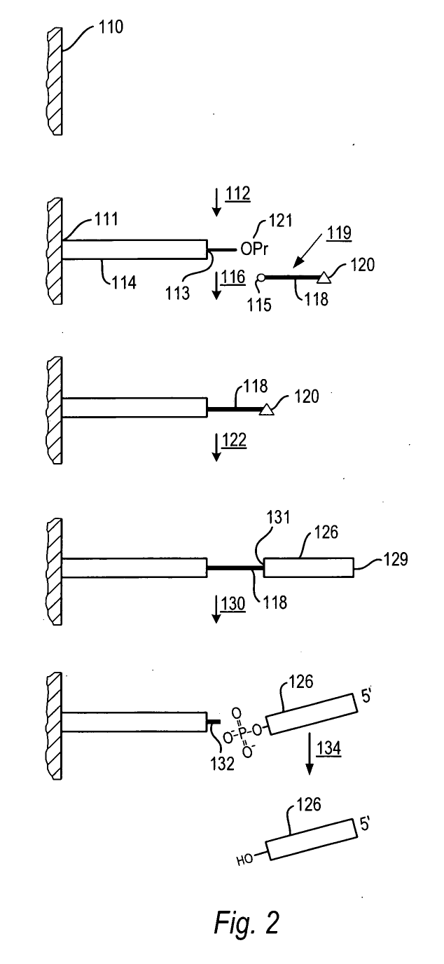

FIG. 2 shows a representative nucleic acid molecule 114 synthesized on a substrate 110 in accordance with some embodiments of the present methods (although it will be understood that, in the practice of the present methods, numerous nucleic acid molecules 114 are simultaneously synthesized on substrate 110). At step 112, proximal nucleic acid molecule 114 is synthesized, as described herein, on substrate 110. Nucleic acid molecule 114 includes a 5′ end 113 and a 3′ end 111 which is covalently attached to the substrate. Step 116 includes removal of hydroxyl protecting group 121, and the resulting 5′ terminal hydroxyl of nucleic acid molecule 114 is then reacted with phosphoramidite group 115 of a cleavable phosphoramidite building block 119, as described herein, to generate a linkage 118. Building block 119 includes protected hydroxyl group 120. At step 122, distal nucleic acid molecule 126 is synthesized, according to methods described herein, and includes a 3′ end 131, and a 5′ end 129. At step 130, the substrate is exposed to conditions that effect cleavage of linker 118 with no release, or essentially no release, of nucleic acid 114 from the substrate 110. In the embodiment shown, a 3′ terminal phosphate is generated in nucleic acid molecule 126. A portion 132 of linker 118 remains attached to nucleic acid molecule 114. In this embodiment, the cleavage of the cleavable linker yields a nucleic acid bearing a phosphate group at the 3′ end. At step 134, the 3′-phosphate end is converted to a 3′-hydroxyl end by a treatment with a chemical or an enzyme (such as alkaline phosphatase) which can be routinely carried out by those skilled in the art.

Multiple nucleic acids of the same or different sequence, linked end-to-end in tandem, can be synthesized by further incorporation of cleavable building block, and nucleic acid synthesis (not shown) prior to cleavage step 30.

In some embodiments, each of the proximal nucleic acid molecules within a defined area (i.e., feature) has a nucleic acid sequence that is essentially identical to the nucleic acid sequence of every other proximal nucleic acid molecule localized to the same defined area. In some embodiments, the proximal nucleic acids of a given feature on the array are made up of single-stranded nucleic acids. In some embodiments, all of the bases are the same (such as a poly T or a poly A nucleic acid). In some embodiments, all of the surface-bound proximal nucleic acids all have the same sequence or different sequences.

The proximal nucleic acid can be attached to the substrate surface by any suitable attachment linkage and the attachment linkage can be selected to remain intact during synthesis, deprotection and cleavage steps, as described. In some embodiments, the attachment linker is selected such that it has a chemistry that is orthogonal to the chemistry used in the cleavable linker. Conditions for cleaving a cleavable linker to selectively release distal nucleic acids as described herein can be readily determined by those skilled in the art, from consideration of the chemistry of the attachment linker and of the cleavable linker. The proximal nucleic acids may be attached to the surface either with or without an intermediate linkage, and may be attached by a non-cleavable attachment linkage as further described herein. As non-limiting examples, in some embodiments, an attachment linker may be photocleavable, while the cleavable linker is acid- or base-labile; in some embodiments, an attachment linker may be acid-labile, while the cleavable linker is base-labile. In some embodiments, the proximal nucleic acid is attached by a linkage which lacks a cleavable moiety.

The proximal nucleic acid may be oriented such that either the 3′ or 5′ end of the molecule is proximal to the substrate surface, e.g., by controlling the synthesis reaction. Exemplary chain lengths of the synthesized proximal nucleic acid molecules can be in the range of about 2 to about 15 nt in length, 1 nt to about 200 nt in length, about 2 to about 100 nt in length, about 2 to about 1000, or more, nt in length.

The distal nucleic acids on a precursor array (i.e., an array prior to cleavage of the cleavable linker) have sequences that are chosen based on the particular application in which the array is to be used, and specifically the intended use of nucleic acids that are released from the array substrate. The length of the distal nucleic acid may vary considerably, and in some embodiments, ranges from about 15 to about 200 nt (nucleotides), from about 20 to about 150 nt, from about 5 to about 500 nucleotides, from about 10 to 10,000, and from about 10 to 1000 nt. In some embodiments, the length of the distal nucleic acid may be at least 10, 50, 100, 1000 nt or more.

In some embodiments, in the practice of the present methods, each of the distal synthesized nucleic acid molecules within a defined area has a nucleic acid sequence that is essentially identical to the nucleic acid sequence of every other distal synthesized nucleic acid molecule localized to the same defined area. In these embodiments, the nucleic acid sequence of the distal nucleic acid molecules in each defined area may be the same as, or different from, the nucleic acid sequence(s) of the distal nucleic acid molecules localized in one or more other defined areas on the substrate. Thus, distal nucleic acid molecules having the same nucleic acid sequence can be synthesized on numerous defined areas of a substrate, thereby providing a large number of distal nucleic acid molecules having the same nucleic acid sequence. In some embodiments, the distal nucleic acids of a given feature on the array are made up of single-stranded nucleic acids.

For example, in some embodiments in which each of the synthesized distal nucleic acid molecules within a defined area has a nucleic acid sequence that is essentially identical to the nucleic acid sequence of every other distal synthesized nucleic acid molecule localized to the same defined area, more than 50% of the defined areas on the substrate contain distal synthesized nucleic acid molecules that have a nucleic acid sequence that is different from the nucleic acid sequences of the distal nucleic acid molecules contained on the other defined areas of the substrate. In some embodiments, greater than 60%, or greater than 70%, or greater than 80%, or greater than 90%, or greater than 95%, or greater than 99%, or all, of the defined areas on the substrate contain distal nucleic acid molecules with a nucleic acid sequence that is different from the sequences of the distal nucleic acid molecules on the other defined areas of the substrate.

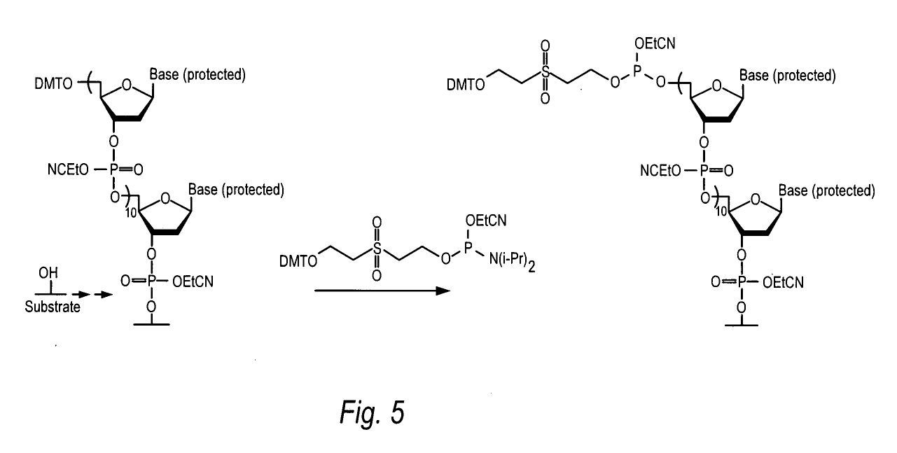

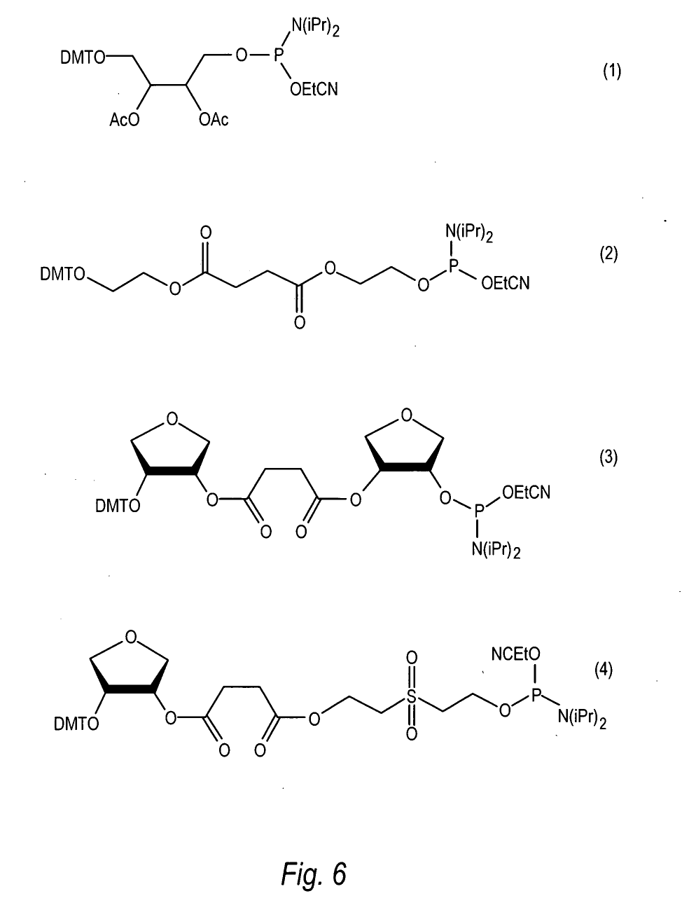

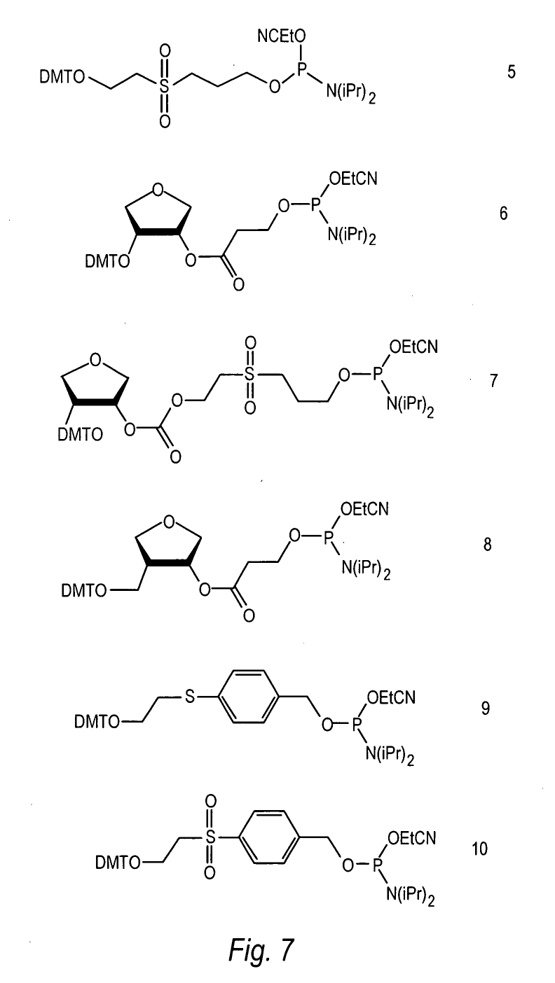

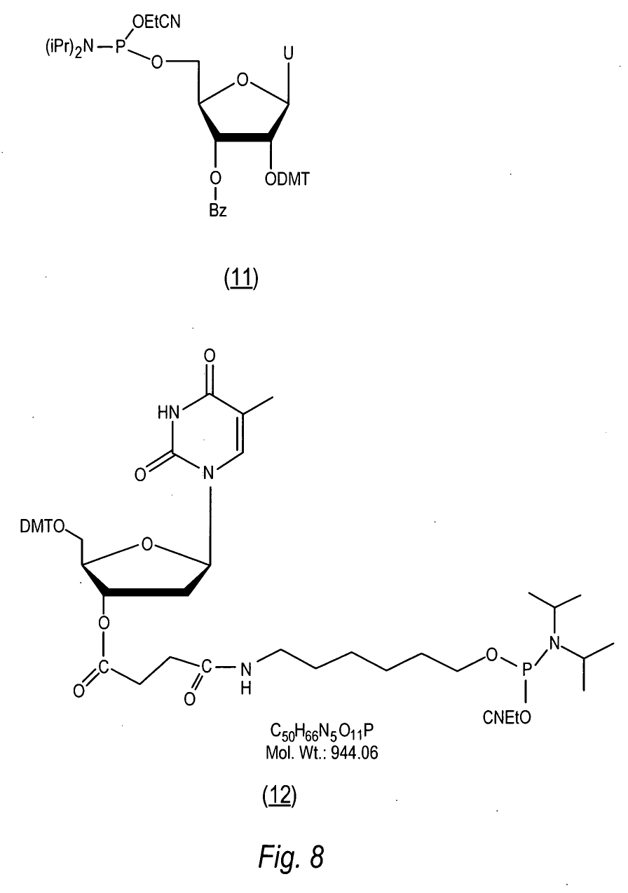

Non-limiting examples of suitable cleavable activated phosphoramidite building blocks include structures 1-12 as shown in FIGS. 4, 6, 7 and 8 (for these and other structures, see, e.g., Hardy et al. (1994) Nucleic Acids Res. 22:2998-3004; Pon et al. (2005) Nucleic Acids Res. 33:1940-1948; Published U.S. Pat. Application Nos. 20030036066; 20030129593; 20040152905; 20050182241; U.S. Pat. Nos. 5,393,877; 5,830,655; 5,869,696; 6,590,002). Structure 12 is available from ChemGenes (catalogue no. CLP-2244 (Thymidine-succinyl hexamide CED phosphoramidite)).

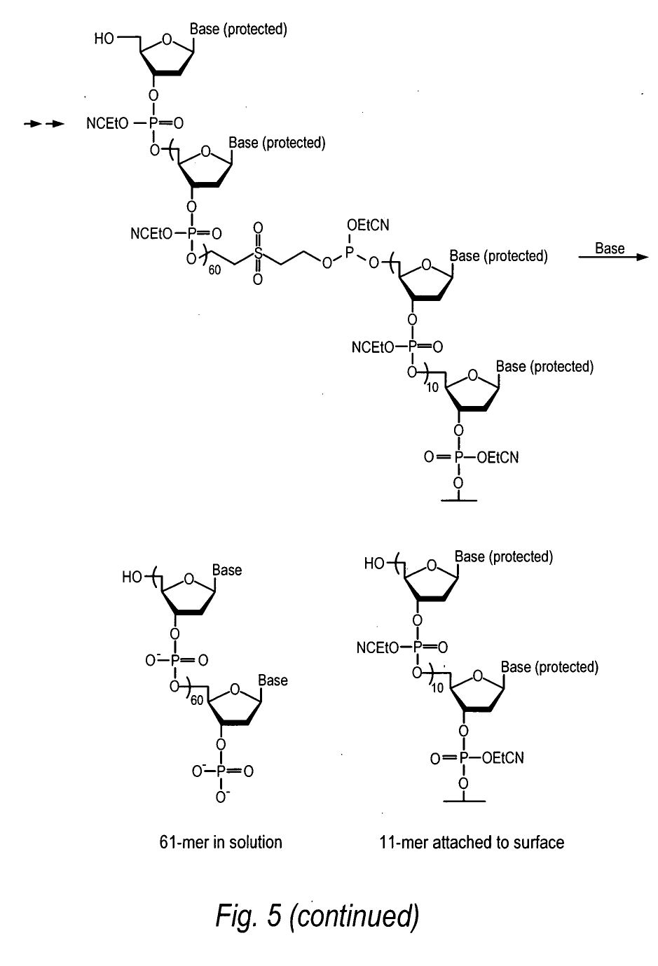

Some embodiments of the synthesis of a nucleic acid incorporating a cleavable linker are shown in FIG. 5. As shown, the distal 61-mer nucleic acid is released upon treatment with base, whereas the proximal 11-mer nucleic acid remains bound to the substrate surface.

Also provided are kits for use in practicing the subject methods. In some embodiments, the kits include one or more of the following: a solid support, an array comprising proximal nucleic acids, an array comprising proximal nucleic acids which have been reacted with a cleavable phosphoramidite building block, a precursor array comprising proximal and distal nucleic acids as described herein, a cleavage reagent for releasing distal nucleic acids from an array, a cleavable phosphoramidite building block, a nucleoside monomer, and a deprotection reagent. Depending on the particular application in which the kits are to be employed, the kits may further include additional containers, each with one or more of the various reagents (e.g., in concentrated form) utilized in specific applications.

A set of instructions may be included, where the instructions may be associated with a package insert and/or the packaging of the kit or the components thereof. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, etc., on which the information has been recorded. Yet another means that may be present is a website address which may be used via the internet to access the information at a removed site.

In some embodiments, the subject methods can include a step of transmitting data (such as, e.g., sequence information related to proximal and/or distal nucleic acids, a precursor array, or a mixture of nucleic acid molecules) to a remote location. For example, a remote location could be another location (e.g. office, lab, etc.) in the same city, another location in a different city, another location in a different state, another location in a different country, etc. As such, when one item is indicated as being “remote” from another, what is meant is that the two items are at least in different buildings, and may be at least one mile, ten miles, or at least one hundred miles apart.

“Communicating” information means transmitting the data representing that information as electrical signals over a suitable communication channel (for example, a private or public network). “Forwarding” an item refers to any means of getting that item from one location to the next, whether by physically transporting that item or otherwise (where that is possible) and includes, at least in the case of data, physically transporting a medium carrying the data or communicating the data. The data may be transmitted to the remote location for further evaluation and/or use. Any convenient telecommunications means may be employed for transmitting the data, e.g., facsimile, modem, internet, etc.

When one item is indicated as being “remote” from another, this descriptor indicates that the two items are in different physical locations, for example, in different buildings, and may be at least about one mile, ten miles, or at least one hundred miles apart. However, in certain aspects, when different items are indicated as being “local” to each other they are not remote from one another (for example, they can be in the same building or the same room of a building). “Communicating”, “transmitting” and the like, of information reference conveying data representing information as electrical or optical signals over a suitable communication channel (for example, a private or public network, wired, optical fiber, wireless radio or satellite, or otherwise). Any communication or transmission can be between devices that are local or remote from one another.

“Forwarding” an item or “providing an item” refers to any means of getting that item from one location to the next, whether by physically transporting that item or using other known methods (where that is possible) and includes, at least in the case of data, physically transporting a medium carrying the data or communicating the data over a communication channel (including electrical, optical, or wireless). “Receiving” something or “being provided” something means, an article/composition/manufacture/data is obtained by any possible means, such as delivery of a physical item (for example, an array or array carrying package). When information is received it may be obtained as data as a result of a transmission (such as by electrical or optical signals over any communication channel of a type mentioned herein), or it may be obtained as electrical or optical signals from reading some other medium (such as a magnetic, optical, or solid state storage device) carrying the information. However, when information is received from a communication it is received as a result of a transmission of that information from elsewhere (local or remote).

A “package” is one or more items (such as an array assembly optionally with other items) all held together (such as by a common wrapping or protective cover or binding). Normally the common wrapping will also be a protective cover (such as a common wrapping or box), which will provide additional protection to items contained in the package from exposure to the external environment. In the case of just a single array assembly a package may be that array assembly with some protective covering over the array assembly (which protective cover may or may not be an additional part of the array unit itself).

In some embodiments, after manufacturing or after obtaining an array from a manufacturer, the array can be subjected to cleavage conditions sufficient to selectively cleave or otherwise release the distal nucleic acids of features on the array to produce a population of nucleic acids. In some embodiments, product plurality of nucleic acids can be shipped or otherwise provided to a user who is remote from the manufacturing site. In some embodiments, the array is shipped and the distal nucleic acids are released from the array at a site remote from the manufacturing site.

When two items are “associated” with one another they are provided in such a way that it is apparent one is related to the other such as where one references the other. For example, an array identifier can be associated with an array by being on the array assembly (such as on the substrate or a housing) that carries the array or on or in a package or kit carrying the array assembly. Items of data are “linked” to one another in a memory when a same data input (for example, filename or directory name or search term) retrieves those items (in a same file or not) or an input of one or more of the linked items retrieves one or more of the others. In particular, when an array layout is “linked” with an identifier for that array, then an input of the identifier into a processor which accesses a memory carrying the linked array layout retrieves the array layout for that array.

A “computer”, “processor” or “processing unit” are used interchangeably and each references any hardware or hardware/software combination which can control components as required to execute recited steps. For example a computer, processor, or processor unit includes a general purpose digital microprocessor suitably programmed to perform all of the steps required of it, or any hardware or hardware/software combination, which will perform those, or equivalent steps. Programming may be accomplished, for example, from a computer readable medium carrying necessary program code (such as a portable storage medium) or by communication from a remote location (such as through a communication channel).

A “memory” or “memory unit” refers to any device that can store information for retrieval as signals by a processor, and may include magnetic or optical devices (such as a hard disk, floppy disk, CD, or DVD), or solid state memory devices (such as volatile or non-volatile RAM). A memory or memory unit may have more than one physical memory device of the same or different types (for example, a memory may have multiple memory devices such as multiple hard drives or multiple solid state memory devices or some combination of hard drives and solid state memory devices).

The subject methods of producing product molecules using a precursor array as described herein find use in a variety of different applications.

In some embodiments, the harvested distal nucleic acid molecules can be amplified. Amplification can be achieved using any method of nucleic acid molecule amplification, including, for example, polymerase chain reaction (PCR), ligase chain reaction (Wu and Wallace, Genomics (1989) 4:560-569; Landegren et al., Science (1988) 241:1077-1080), transcription amplification (Kwoh et al., Proc. Nat'l. Acad. Sci. (1990) 87:1874-1878), self-sustained sequenced replication (Guantelli et al. (1987) Proc. Nat'l. Acad. Sci. 87:1874-1878), and nucleic acid based sequence amplification (NASBA).

PCR amplification methods are well known in the art and are described, for example, in Innis et al., eds. (1990) PCR Protocols: A Guide to Methods and Applications, Academic Press Inc. San Diego, Calif. An amplification reaction typically includes the DNA that is to be amplified, a thermostable DNA polymerase, two oligonucleotide primers, deoxynucleotide triphosphates (dNTPs), reaction buffer and magnesium. Typically a desirable number of thermal cycles is between 1 and 25. Methods for primer design and optimization of PCR conditions are well known in the art and can be found in standard molecular biology texts such as Ausubel et al. (1995) Short Protocols in Molecular Biology, Wiley; and Innis et al. (1990) PCR Protocols, Academic Press. Taq DNA polymerase generates single dA overhangs on the 3′ ends of the PCR product, allowing for ease of cloning into vectors that contain “T” overhangs complementary to those on the PCR product, such as TA Cloning vectors (available from Invitrogen Corporation, 1600 Faraday Avenue, P.O. Box 6482, Carlsbad, Calif. 92008).

Any primers that are complementary to a portion of the distal nucleic acid molecules that are synthesized on the substrate can be used to prime the polymerase chain reaction. For example, in some embodiments, a primer hybridizes to a 5′ primer binding region of the distal nucleic acid molecule to be amplified, and the same primer, or a different primer, hybridizes to a 3′ primer binding region of the distal nucleic acid molecule to be amplified. The primer binding regions of the distal nucleic acid molecules to be amplified, and hence the corresponding complementary PCR primers, can range in length from about 4 to about 30 nucleotides. Computer programs are useful in the design of primers with the required specificity and optimal amplification properties (e.g., Oligo Version 5.0 (National Biosciences)). In some embodiments, the PCR primers may additionally contain recognition sites for restriction endonucleases, to facilitate insertion of the amplified DNA fragment into specific restriction enzyme sites in a vector. If restriction sites are to be added to the 5′ end of the PCR primers, it is preferable to include a few (e.g., two or three) extra 5′ bases to allow more efficient cleavage by the enzyme. In some embodiments, the PCR primers may also contain an RNA polymerase promoter site, such as T7 or SP6, to allow for subsequent in vitro transcription in order to create a library of RNA molecules derived from the nucleic acid molecules that were synthesized on the substrate.

PCR amplification products can be purified using any suitable means. For example, such means include gel electrophoresis, column chromatography, high pressure liquid chromatography (HPLC) or physical means such as mass spectroscopy.

In some embodiments, once synthesized distal nucleic acid molecules are harvested they can be cloned into vector molecules. Typically, harvested distal nucleic acid molecules are single stranded DNA molecules which may require second-strand synthesis to form double stranded DNA molecules prior to cloning into vector molecules. Second-strand synthesis may be achieved, for example, by first annealing a DNA oligonucleotide primer to a portion of each of the released distal nucleic acid molecules (e.g., annealing a primer that hybridizes to a primer binding region). A DNA polymerizing enzyme, such as Taq polymerase or the Klenow fragment of E. coli DNA polymerase I, can then added to complete second-strand synthesis, resulting in double-stranded DNA molecules. Second strand synthesis can also occur, for example, during the first cycle of a series of amplification reactions (e.g., PCR reactions).

In some embodiments, distal synthesized nucleic acid molecules can be harvested from a substrate, and then introduced into vector molecules to form a nucleic acid library (see, e.g., US Pat. Publication 20040259146). The term “vector” refers to a nucleic acid molecule, usually double-stranded DNA, which is designed to receive another nucleic acid molecule (usually called the insert nucleic acid molecule), such as a distal nucleic acid molecule synthesized in accordance with the present methods. The vector is typically used to transport the insert nucleic acid molecule into a suitable host cell, or can be used, for example, in an in vitro system capable of utilizing elements in the vector. A vector may contain the necessary elements that permit transcribing, and optionally translating, the insert nucleic acid molecule into an RNA molecule, and optionally a polypeptide. This type of vector is called an expression vector. The insert nucleic acid molecule can be any nucleic acid molecule. Once in the host cell, the vector may replicate independently of, or coincidental with (e.g., by genomic integration), the host chromosomal DNA, and several copies of the vector and its inserted nucleic acid molecule may be generated.

Vectors useful in the practice of some embodiments of the present methods can also include other regulatory sequences, such as promoters, translation leader sequences, introns, and polyadenylation signal sequences. “Promoter” refers to a DNA sequence involved in controlling the expression of a coding sequence or functional RNA. In general, a coding sequence is located within the molecule at a position 3′ of the promoter sequence. The term “promoter” includes a minimal promoter that is a short DNA sequence comprised of a TATA-box and/or other sequences that serve to specify the site of transcription initiation, to which regulatory elements may be added for control of expression. Promoters may be derived in their entirety from a native gene, or be composed of different elements derived from different promoters found in nature, or even comprise synthetic DNA segments.

Examples of vectors include plasmids, phages, cosmids, phagemids, viruses (e.g., retroviruses, lentiviruses, parainfluenzavirus, herpesviruses, reoviruses, paramyxoviruses, and the like). Commonly, vectors contain selection markers, such as genes encoding drug resistance to tetracycline, neomycin, hygromycin, or puromycin, or other genes that permit selection of cells transduced with the desired DNA sequences, such as hypoxanthine guanine phosphoribosyl transferase (HPRT), dihydrofolate reductase (DHFR), or thymidine kinase (TK).

Examples of vectors that are functional in plants are binary plasmids derived from Agrobacterium plasmids. Such vectors are capable of genetically transforming plant cells. Briefly, these vectors typically contain left and right border sequences that are required for integration into the host (plant) chromosome. Typically, between these border sequences is the nucleic acid molecule (such as a cDNA) to be expressed under control of a promoter. In some embodiments, a selectable marker and a reporter gene are also included. The vector also may contain a bacterial origin of replication.

Methods for introducing DNA inserts into vectors are well known in the art (see Sambrook et al. (1989) Molecular Cloning: A Laboratory Manual, 2^{nd} ed., Cold Spring Harbor Press, Plainview, N.Y., and Ausubel et al. (1999) Current Protocols in Molecular Biology (Supplement 47), John Wiley & Sons, New York). Various methods can be used in the cloning process, such as for example, PCR products that have restriction enzyme sites incorporated within, either as a result of synthesis or as a consequence of PCR amplification utilizing primers containing such sites, can be digested and cloned into a plasmid vector with compatible ends. Alternatively, selective adaptors having recognition sites compatible with the expression vector of choice can be ligated to the ends of PCR products. Selective adaptors can be produced by well-known methods for the production of oligonucleotides (see Gait (1984) Oligonucleotide Synthesis: A Practical Approach, IRL Press). Double stranded adaptors are typically produced one strand at a time and annealed prior to addition to the digested insert population. Adaptors can also be added to the ends of amplification primers. In addition, TA cloning vectors (Invitrogen) which contain 3′ T overhangs can be used to clone PCR products that have been amplified using Taq polymerase and therefore have a corresponding 3′ A overhang on the end of each PCR product.

The vectors containing the DNA inserts of interest may be transferred into a host cell by well-known methods, depending on the type of cellular host. For example, calcium chloride transfection is commonly utilized for prokaryotic cells, whereas calcium phosphate treatment, lipofection or electroporation are exemplary procedures that may be used for other cellular hosts. Other methods used to transform mammalian cells include the use of viral infection, polybrene, protoplast fusion, liposomes, cationic transfection procedures, and microinjection. Once the vector has been incorporated into an appropriate host, the host may be maintained under conditions suitable for high level expression of the nucleotide sequences, and expressed polypeptides collected and purified. Once purified, the polypeptides can be used, for example, in screening assays.

The overall quality of the nucleic acid molecule synthesis can be assessed at several stages during practice of the present methods. For example, the quality of nucleic acid synthesis can be determined prior to harvesting the distal nucleic acid molecules from the substrate, using functional hybridization of a standard quality control template.

In some embodiments, to facilitate amplification of the distal nucleic acid molecules, each distal nucleic acid molecule may include a 5′ primer binding region, and a 3′ primer binding region. In these embodiments, the portion of the nucleic acid molecule located between the 5′ primer binding region and the 3′ primer binding region is referred to as the target sequence.

In some embodiments, to facilitate amplification and cloning of the distal nucleic acid molecules into a vector, each synthesized nucleic acid molecule may include a 5′ primer binding region, and a 3′ primer binding region. In these embodiments, the portion of the nucleic acid molecule located between the 5′ primer binding region and the 3′ primer binding region is referred to as the target sequence. The target sequence may, for example, encode a portion of a protein that is to be expressed.

In some embodiments, distal nucleic acid molecules further comprise an RNA polymerase promoter site, such as T7 or SP6, to allow for subsequent, in vitro, transcription in order to create a library of RNA molecules derived from the distal nucleic acid molecules.

In some embodiments, the 5′ primer binding region and the 3′ primer binding region of the distal nucleic acid molecules range in length from, e.g., about 4 to about 1000, from 5 to 500, or from 10 to 200 nucleotides, and may include restriction enzyme cleavage sites. The nucleotide sequences of the 5′ binding region and 3′ primer binding region may be chosen to allow for efficient amplification and may have an annealing temperature within about 20° C. of each other. Computer programs are useful in the design of primers with the required specificity and optimal amplification properties. See, e.g., Oligo version 5.0 (available from National Biosciences Inc., 3001 Harbor Lane, Suite 156, Plymouth, Minn. 55447). The same 5′ primer binding region and/or 3′ primer binding region may be present in all of the distal nucleic acid molecules, or a particular 5′ primer binding sequence or 3′ primer binding sequence may be present in only a subpopulation of the distal nucleic acid molecules, thereby allowing for selective amplification of the subpopulation of the distal nucleic acid molecules. Target sequences of the distal nucleic acid molecules may encode, for example, a portion of a protein to be expressed. In some embodiments, the target sequence of each distal nucleic acid molecule localized to a particular defined area of the substrate is different from the target sequence of each distal nucleic acid molecule localized to different defined areas of the substrate. Thus, in some embodiments, each defined area on a substrate contains a different target sequence. In some embodiments, more than 50% of the defined areas on the substrate contain distal nucleic acid molecules that have a target sequence that is different from the target sequences of the nucleic acid molecules contained on the other defined areas of the substrate. In some embodiments, greater than 60%, or greater than 70%, or greater than 80%, or greater than 90%, or greater than 95%, or greater than 99%, or all, of the defined areas on the substrate contain distal nucleic acid molecules with a target sequence that is different from the sequence of all of the target sequences on separate defined areas of the substrate.

In some embodiments, the distal nucleic acid molecules additionally contain a target identifier sequence to facilitate selective amplification of a particular target sequence out of the population of distal nucleic acid molecules. Typically, the length of the target identifier sequence is from about 4 base pairs to about 8 base pairs. The target identifier sequence can be located anywhere within the distal nucleic acid molecule, such as immediately adjacent to either the 5′ end or the 3′ end of the distal nucleic acid molecule. A target identifier sequence that consists of only four bases provides for 256 different unique nucleic acid sequences, and a target identifier sequence that consists of only eight bases provides for 65,536 different unique nucleic acid sequences. In some embodiments, each target identifier sequence is associated with a particular target sequence. In some embodiments, each target identifier sequence is associated with a predetermined sub-population of target sequence(s).

In some embodiments, a mixture can comprise a population of nucleic acid molecules. An art-recognized term for a population of nucleic acid molecules is a “library” of nucleic acid molecules. The term “library” is usually, although not necessarily, applied to populations of nucleic acid molecules that have been introduced into vector molecules that facilitate expression of the nucleic acid molecules to yield other nucleic acid molecules (e.g., RNA molecules) and/or proteins (or fragments of complete proteins). For example, the methods of the disclosure can be used to create nucleic acid libraries for antibody diversity studies, phage display, combinatorial peptide sequence generation, DNA binding site selection, promoter structural analysis, identification of regulatory sequences, restriction enzyme recognition site analysis, short hairpin RNA (shRNA) expression, small interfering RNA (siRNA) expression, chromosomal probe generation, genomic insertional mutagenesis, creation of nucleic acid multimers and screening sequences for protein domain solubility in expression systems.

By way of non-limiting example, the methods disclosed herein can be used to generate a nucleic acid library to analyze variations of a protein subdomain, such as, for example, a catalytic domain, activation domain, DNA-binding domain, protein interaction domain, nuclear localization domain, or antibody structural domain. The methods are also useful, for example, for generating libraries expressing random amino acid sequence polypeptide fragments, or for producing random mutagenesis of protein fragments. Such libraries can be designed in various ways so that either the insert alone is expressed, the insert is embedded into a framework of the wild-type, or engineered, protein flanking sequence residing in the vector such that variations of the protein are expressed, the insert is fused to a reporter protein, the insert is tagged with an epitope, or the insert itself can encode an epitope. Such libraries can be expressed, for example, intracellularly in tissue culture, in bacterial cells (e.g., as GST fusions), in animal model systems, in in vitro translation systems (e.g., rabbit reticulocyte lysate), in cell extracts and through phage display.

By way of non-limiting example, the methods of the disclosure can be used to generate a nucleic acid library to analyze the functional relationship between the amino acid sequence and binding specificity of a DNA-binding protein, such as, for example, a zinc finger protein. Zinc finger proteins contain DNA binding motifs (referred to as “fingers”) which typically contain an approximately 30 amino acid, zinc chelating, DNA binding subdomain. (see e.g., Berg & Shi, Science 271:1081-1085 (1996)). The DNA binding affinity of zinc finger proteins can be enhanced through the design and synthesis of a preselected population of sequence variations of the DNA binding subdomain, such as a sequential substitution of each nucleic acid residue in the DNA binding subdomain. Once synthesized, the population of nucleic acid molecules containing sequence variations of the DNA binding motif can be cloned into a vector to form a library, which can be introduced into host cells and expressed therein. The polypeptides encoded by the library of predetermined nucleic acid molecules can then be screened for the desired properties, such as, for example, enhanced DNA binding affinity. In the case of a DNA binding protein whose recognition sequence is not known, the methods of the disclosure can be used to generate a nucleic acid library containing random sequences to enable selection of the sequence with the highest affinity for the DNA binding site. An example of such an approach is a yeast one-hybrid system, in which the fusion protein remains constant and the DNA recognition sequence driving expression of the reporter construct contains random sequences which are selected based on expression of the reporter gene.

Again by way of non-limiting example, the disclosed methods can be used to generate cassettes for genomic insertional mutagenesis. For example, synthesized nucleic acid molecules containing sequences homologous to a specific genomic locus can be cloned into a targeting construct to allow for homologous recombination and disruption of a specific genomic region.

By way of further example, the disclosed methods can be used to produce multimers of specific sequences. To produce such multimers, single stranded nucleic acid molecules are synthesized in accordance with the present disclosure, and then rendered double stranded (e.g., by annealing complementary single stranded nucleic acid molecules). Individual, double-stranded, nucleic acid molecules can be joined using a DNA ligase. Multimers of a desired size can be selected prior to cloning.

In another exemplary use, the disclosed methods can be used for testing protein domain solubility in bacteria. This can be achieved, for example, by fusing the synthesized nucleic acid molecules to the coding region of green fluorescent protein (GFP) in a bacterial protein expression plasmid, and screening for fluorescence in bacteria.

By way of non-limiting example, the disclosed methods can be used to generate a library expressing variations of functional RNAs (e.g., short hairpin RNAs, short interfering RNAs, ribozymes, small nuclear RNAs, small nucleolar RNAs, transfer RNAs, small temporal RNAs, etc). Such libraries can be designed so that either the insert alone is expressed, the insert is embedded into a framework of wild-type RNA flanking sequence, or the insert is fused to a reporter gene (e.g., luciferase, GFP). Such libraries can be expressed, for example, in vitro, in bacterial cells, mammalian cells or in animal model systems.

Again by way of non-limiting example, the disclosed methods can be used to make a phage display library using a phage DNA vector from which is transcribed a fusion protein, a portion of which is encoded by an insert nucleic acid molecule introduced into the vector. Phage display libraries are useful, for example to isolate antibody fragments (e.g., Fab, Fv, scFv and VH) based on antibody specificity to a particular antigen. A phage containing an insert nucleic acid molecule undergoes replication and transcription in the cell to yield a fusion protein. The leader sequence of the fusion protein directs the transport of the fusion protein to the tip of the phage particle. Thus, the fusion protein which is partially encoded by the insert nucleic acid molecule is displayed on the phage particle for detection and selection.

By way of further example, the disclosed methods can be used to make a peptide display library. One exemplary peptide display method involves the presentation of a peptide sequence on the surface of a filamentous bacteriophage, typically as a fusion with a bacteriophage coat protein. The bacteriophage library can be incubated with an immobilized, predetermined macromolecule or small molecule (e.g., a receptor) so that bacteriophage particles which present a peptide sequence that binds to the immobilized macromolecule can be differentially partitioned from those that do not present peptide sequences that bind to the predetermined macromolecule. The bacteriophage particles that are bound to the immobilized macromolecule are then recovered and replicated to amplify the selected bacteriophage sub-population for a subsequent round of affinity enrichment and phage replication. After several rounds of affinity enrichment and phage replication, the bacteriophage library members that are thus selected are isolated and the nucleotide sequence encoding the displayed peptide sequence is determined, thereby identifying the sequence(s) of peptides that bind to the predetermined macromolecule (e.g., receptor). Such peptide display methods are further described, for example, in PCT Pat. Application Nos. 91/17271, 91/18980, 91/19818 and 93/08278.

It is noted that the above reviewed nucleic acid applications are merely representative of the diverse types of applications in which the subject methods find use, and that the subject methods are not limited to use merely in the above representative applications.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A method for synthesizing nucleic acid molecules, said method comprising the steps of:
a) synthesizing an array of proximal nucleic acid molecules on a substrate,
b) incorporating a cleavable linker by contacting the array of proximal nucleic acid molecules with a cleavable phosphoramidite building block comprising:
R-Lc-Pr
wherein Pr is a hydroxyl protecting group,
Lc is a cleavable linker, and
R is a phosphoramidite group,
c) extending the building block to form distal nucleic acid molecules, and
d) cleaving the cleavable linker to release the distal nucleic acid molecules under conditions which do not release the proximal nucleic acid molecules.

2. The substrate of claim 1 wherein the proximal nucleic acid molecules are bound to the surface by an attachment linkage, and wherein the attachment linkage is chemically orthogonal to the cleavable linker.

3. The substrate of claim 1 wherein the proximal nucleic acid molecules are bound to the surface by an attachment linkage, and wherein the attachment linkage is devoid of a cleavable moiety.

4. The substrate of claim 1 wherein the proximal nucleic acid molecules are bound to the surface by an attachment linkage, and wherein the attachment linkage is devoid of an ester.

5. The substrate of claim 1 wherein the proximal nucleic acid molecules are bound to the surface by an attachment linkage, and wherein the attachment linkage comprises a non-cleavable linkage.

6. The method of claim 1, wherein the proximal nucleic acid molecules are 2 to 30 nucleotide residues in length.

7. The method of claim 1, wherein the distal nucleic acid molecules are 10 to 500 nucleotide residues in length.

8. The method of claim 1, wherein the proximal nucleic acid molecules comprise the same base.

9. The method of claim 1 comprising deprotecting all of the distal nucleic acid molecules after step (c).

10. The method of claim 1, wherein each of the distal nucleic acid molecules released in step (d) comprises a 3′ terminal hydroxyl group.

11. The method of claim 1, wherein the cleavable linker is susceptible to cleavage with base.

12. The method of claim 1, wherein the cleavable linker is susceptible to cleavage with an enzyme.

13. The method of claim 1, wherein each of the distal nucleic acid molecules released in step (d) comprises a 3′phosphate group, and wherein the method comprises removing 3′phosphate group.

14. The method of claim 1, wherein said substrate comprises a non-porous glass surface.

15. The method of claim 1, comprising introducing released distal nucleic acid molecules into vector molecules.

16. The method of claim 1, wherein the method comprises using a pulse jet to deposit reagents at each of a plurality of sites in the array.

17. The method of claim 1, wherein step (d) comprises contacting the surface with a cleavage agent effective to cleave the cleavable linker, said contacting being for a time and under conditions sufficient to result in cleaving the cleavable linker.

18. The method of claim 17, said conditions also being sufficient to concurrently deprotect the distal nucleic acids.

19. The method of claim 17, comprising, prior to contacting the surface with the cleavage agent, contacting the array with a deprotection agent.

20. The method of claim 1, comprising recovering a solution phase mixture comprising the distal nucleic acids.

21. The method of claim 1, wherein the cleavable linker includes a cleavable moiety selected from a photocleavable moiety and a chemically cleavable moiety.

22. The method of claim 1, wherein the cleavable linker comprises a chemically cleavable moiety selected from an acid-cleavable moiety, a base-cleavable moiety, and a nucleophile-cleavable moiety.

23. A method for preparing a nucleic acid, the method comprising the steps of:
a) providing a solid support comprising:
sm-PN₁-Lc-PN₂,
wherein sm is a substrate medium,
wherein PN₁ is non-cleavably attached to the support,
wherein Lc comprises a cleavable linker, and
b) selectively releasing said PN₂.

24. A method for the synthesis of a plurality of oligonucleotides comprising the steps of
(i) forming an array of first oligonucleotides on a non-cleavable link attached to a solid support;
(ii) attaching to the first oligonucleotides a cleavable linker moiety;
(iii) forming second oligonucleotides on the cleavable linker moiety; and
(iv) cleaving the cleavable linker moiety to give a plurality of oligonucleotides,
wherein the first oligonucleotides are bound to the surface by an attachment linkage, and wherein the attachment linkage is devoid of a cleavable moiety.

25. A method according to claim 24, wherein cleavage of the cleavable linker moiety results in a plurality of oligonucleotides each having a hydroxy at the 3′ position.

26. A composition comprising:
a modified substrate medium according to the following formula:
sm-PN₁-Lc-PN₂
wherein sm is a substrate medium,
wherein Lc comprises a cleavable linker,
wherein PN₁ is a polynucleotide from 2-100 residues in length,
wherein PN₂ is a polynucleotide from 5 to 1000 residues in length,
wherein PN₁ is attached to the substrate medium by a non-cleavable attachment.

27. The composition of claim 26, wherein all of the residues of PN₁ are the same.

28. The composition of claim 26, wherein the PN₁ and the PN₂ comprise fully protected bases.

29. The composition of claim 26, wherein said substrate medium comprises a planar medium and a plurality of features arranged in an array, wherein each feature comprises a plurality of identical PN₂ molecules.

30. The composition of claim 26, wherein said substrate medium comprises a planar medium.

31. The composition of claim 26, wherein said substrate comprises a bead.

32. A composition comprising:
a modified substrate medium according to the following formula:
sm-PN₁-Lc-Pr
wherein sm is a substrate medium,
wherein Pr is a hydroxyl protecting group,
wherein Lc comprises a cleavable linker,
wherein PN₁ is a polynucleotide from 2-100 residues in length,
wherein said PN₁ is bound to the surface by an attachment linkage means, and wherein
the attachment means is devoid of cleavable moieties.

33. A compound of formula I:
sm-PN₁-Lc-PN₂ I
wherein sm is a substrate medium,
wherein Lc comprises a cleavable linker,
wherein PN₁ is a polynucleotide from 2-100 residues in length,
wherein PN₂ is a polynucleotide from 5 to 1000 residues in length,
wherein said sm is coupled to said PN₁ by a non-cleavable linker, and said PN₁ is coupled to said PN₂ by a cleavable linker.

34. A kit for preparing a mixture of nucleic acids, comprising:
a) a modified substrate medium according to the following formula:
sm-PN₁-Lc-PN₂
wherein sm is a substrate medium,
wherein Lc comprises a cleavable linker,
wherein PN₁ is a polynucleotide from 2-100 residues in length,
wherein PN₂ is a polynucleotide from 5 to 1000 residues in length,
wherein PN₁ is attached to the substrate medium by a non-cleavable linkage, and
b) a cleavage agent capable of cleaving said cleavable linker.

35. A kit for preparing a mixture of nucleic acids, comprising:
a) a cleavable phosphoramidite building block comprising a cleavable linker,
b) a cleavage agent for cleaving said cleavable linker,
c) a modified substrate medium according to the following formula:
sm-PN₁-Pr
wherein sm is a substrate medium,
wherein Pr is a hydroxyl protecting group,
wherein PN₁ is bound to the surface by an attachment linkage means, and wherein the attachment linkage is non-cleavable.
